(19) United States
(12) Patent Application Publication
(10) Pub. No.: US 2025/0352610 A1
(43) Pub. Date: Nov. 20, 2025

(54) NOVEL COMPOSITION

(71) Applicant: Arecor Limited, Little Chesterford, Saffron Walden (GB)

(72) Inventors: Andrew HALL, Little Chesterford, Saffron Walden (GB); Azadeh BAHREMAN, Little Chesterford, Saffron Walden (GB); Adrian PODMORE, Little Chesterford, Saffron Walden (GB); Jan JEZEK, Little Chesterford, Saffron Walden (GB)

(21) Appl. No.: 18/866,840

(22) PCT Filed: May 19, 2023
(86) PCT No.: PCT/GB2023/051330

§ 371 Date: Nov. 18, 2024

(30) Foreign Application Priority Data
May 19, 2022 (GB) 2207344.9

(51) Int. Cl. A61K 38/14; A61K 9/00; A61K 47/02; A61K 47/10; A61K 47/12; A61K 47/26

(52) U.S. Cl. CPC A61K 38/14; A61K 9/0019; A61K 47/02; A61K 47/10; A61K 47/12; A61K 47/26

(57) ABSTRACT

There is provided inter alia a storage stable aqueous solution composition comprising vancomycin or a pharmaceutically acceptable salt thereof, at a concentration of 1-10 mg/mL; D-lactic acid or a pharmaceutically acceptable salt thereof; and a polyol selected from the group consisting of propylene glycol, sucrose, glycerol, trehalose, lactose, glucose, sorbitol and mannitol, or a mixture thereof; wherein the osmolarity of the composition is 400-1250 mOsm/L.

This invention relates to aqueous solution compositions of vancomycin, in particular ready-to-administer compositions which are storage stable and non-toxic.

### BACKGROUND

Vancomycin is a glycopeptide antibiotic, consisting of a heptapeptide chain which forms a tricyclic ring system, with sugars attached. The N-terminal amino acid leucine is critical for antibacterial activity. Vancomycin is derived from the fungus Amycolatopsis orientalis (formally Nocardia orientalis). It is used to treat various infections that are caused by gram positive bacteria, such as methicillin-resistant Staphylococcus aureus (MRSA). Vancomycin is typically used in the form of its hydrochloride salt, with molecular weight of 1485.71 Da.

Vancomycin blocks the transpeptidase step of bacterial cell wall synthesis by preventing the synthesis of key polymers within the peptidoglycan layer. Vancomycin forms hydrogen bonds with the D-alanyl-D-alanine moieties of the N-acetylmuramic acid (NAM) and N-acetylglucosamine (NAG) peptides. This binding prevents the synthesis of NAM and NAG peptides that form the backbone strands of the bacterial cell wall.

Vancomycin can be administered either orally or intravenously, depending on specific circumstances. The invention disclosed herein is primarily concerned with intravenously administered vancomycin.

There are a number of vancomycin products for intravenous use that are lyophilised. These products require reconstitution followed by dilution into an intravenous (IV) solution bag and are therefore rather inconvenient to use.

There are three commercial liquid products on the market that comprise pre-mixed formulations of vancomycin formulated in IV solution bags and that can be administered directly: Vancomycin Injection, USP IV Frozen Premix (marketed by Baxter) and VancoREADY™ (marketed by Xellia). For the purpose of the present invention such pre-mixed products filled into IV solution containers such as bags or bottles are referred to as “ready-to-administer” products.

Baxter USP IV premix, formulated in the GALAXY plastic container is a frozen, premixed 100 mL, 150 mL, or 200 mL solution containing 500 mg, 750 mg, or 1 g vancomycin respectively as vancomycin hydrochloride. Each 100 mL of solution contains approximately 5 g of glucose or 0.9 g of sodium chloride. The pH of the solution has been adjusted with hydrochloric acid and/or sodium hydroxide. The product must be stored in a freezer capable of maintaining temperature at or below −20° C. Thawed solutions have a pH in the range of 3.0 to 5.0. After thawing at room temperature (25° C.) or under refrigeration condition (5° C.), this solution is intended for intravenous use only. The need to store in frozen form and the need to thaw before use causes some limitations and inconvenience for medical workers.

VancoREADY™ exists as a liquid product and is supplied in a single dose premixed IV solution bag as a clear and colourless to light brown, sterile, preservative-free solution. It is available in 7 different doses from 500 mg-2 g. Each 100 mL of solution contains 500 mg vancomycin hydrochloride, 1.8 mL PEG400, 1.36 g N-acetyl-D-alanine, 1.26 g L-lysine hydrochloride, at pH 5.0. Long term storage is at room temperature (25° C.) for 16 months in aluminium overwrap, after removal from overwrap it is still stable for a further 28 days at 25° C. VancoReady™ carries a “black box” warning due to the excipients of N-acetyl-D-alanine and PEG400 which have been known to cause fetal malformations in animal reproductive studies, so this product is advised against for pregnant women.

The present invention addresses the need for stable and non-toxic vancomycin compositions.

WO2014/194296A1 (SCIDOSE, LLC.) discloses compositions comprising vancomycin or a pharmaceutically acceptable salt thereof; a polar solvent such as propylene glycol, polyethylene glycol and mixtures thereof; lactic acid, a lactate salt, or mixtures thereof; and optionally, a pH adjuster in an amount sufficient to maintain a pH of the compositions at from about 3 to about 8.

WO2017/123912A1 (SCIDOSE, LLC.) discloses compositions comprising vancomycin or a pharmaceutically acceptable salt thereof, a polyol such as glycerol, and lactic acid or a lactate.

US2020/0188478A1 (FTF PHARMA PRIVATE LIMITED) discloses pre-mixed vancomycin compositions said to be suitable for injection.

JP2008/201778A (MOCHIDA PHARM CO LTD.) discloses vancomycin compositions said to be suitable for long-term storage and oral administration.

WO2017/194385A1 (XELLIA PHARMACEUTICALS APS) discloses liquid formulations of glycopeptide antibiotics such as vancomycin, which are said to be suitable as infusion solutions.

US2018/0133286A1 (XELLIA PHARMACEUTICALS APS) discloses compositions containing vancomycin, and an amino acid or amino acid derivative such as N-acetyl-glycine or N-acetyl-D-alanine.

### SUMMARY OF THE INVENTION

The present invention provides, inter alia, a storage stable aqueous solution composition comprising:
vancomycin or a pharmaceutically acceptable salt thereof, at a concentration of 1-10 mg/mL;
D-lactic acid or a pharmaceutically acceptable salt thereof; and
a polyol selected from the group consisting of propylene glycol, sucrose, glycerol, trehalose, lactose, glucose, sorbitol and mannitol, or a mixture thereof;
   wherein the osmolarity of the composition is 400-1250 mOsm/L.

### DETAILED DESCRIPTION OF THE INVENTION

In the compositions of the invention vancomycin may be present in the form of a pharmaceutically acceptable salt. Examples of pharmaceutically acceptable salts include hydrochloride, citrate, formate, acetate, tartrate, sulphate, tosylate, nitrate, mesylate, napsylate, besylate, oxalate, maleate, phosphate, pamoate, fumarate, hippurate, succinate and carbonate. In one embodiment, the vancomycin form is vancomycin hydrochloride. For the avoidance of doubt, any reference herein to “vancomycin” is also intended to cover a pharmaceutically acceptable salt of vancomycin, in particular the hydrochloride salt, unless stated otherwise.

Compositions of the present invention are “ready-to-administer”, in the sense that they can be administered directly to a patient without dilution. The composition is typically administered intravenously and stored in an IV solution container such as a bag or bottle. In one embodiment, the concentration of vancomycin or pharmaceutically acceptable salt thereof is 1-9 mg/mL, 2-8 mg/mL, 2.5-7.5 mg/mL or about 5 mg/mL.

As described in the background of invention, the currently marketed liquid vancomycin composition VancoReady™ contains N-acetyl-D-alanine (NADA) and PEG400 as excipients, both of which have been linked to fetal malformations in animal reproductive studies, therefore are to be avoided in this context. As described in present Example 3, the N-acetyl-D-alanine (NADA) in the VancoReady™ composition appears to provide a stabilizing effect. As described in Example 4, the present inventors have discovered that using D-lactate in place of NADA, a stable composition is formed. No stabilizing effect was observed when L-lactate was used in place of NADA. As shown in Examples 6 and 7, the racemic form of lactate (DL-lactate) also provides a stabilizing effect, but the concentration is typically twice as high as D-lactate to achieve a comparable stabilizing effect.

In one embodiment, the composition comprises a pharmaceutically acceptable salt of D-lactic acid (also referred to herein as “D-lactate”). Suitable pharmaceutically acceptable salts of D-lactic acid include sodium D-lactate, calcium D-lactate and potassium D-lactate. In one embodiment, the pharmaceutically acceptable salt of D-lactic acid is sodium D-lactate. In one embodiment, the concentration of D-lactic acid or a pharmaceutically acceptable salt thereof is 100-500 mM, 200-500 mM, 100-400 mM, 200-400 mM, 100-300 mM, 200-300 mM, 100-200 mM, 100-150 mM, 150-250 mM, about 200 mM or about 100 mM.

In one embodiment, the composition is substantially free of, or free of L-lactic acid and salts thereof i.e. the only form of lactic acid or lactate in the composition is D-lactic acid or a pharmaceutically acceptable salt thereof. In this embodiment, “substantially free of” means that less than 1% of the molar concentration of total lactic acid (i.e. total molar concentration of D-lactic acid or salts thereof and L-lactic acid or salts thereof) is in the form of L-lactic acid or salts thereof, e.g. for a composition comprising D-lactic acid or a pharmaceutically acceptable salt thereof at a concentration of 500 mM, the composition will contain less than 5 mM of L-lactic acid or a salt thereof.

In one embodiment, the source of D-lactic acid or a pharmaceutically acceptable salt thereof is DL-lactic acid or a pharmaceutically acceptable salt thereof (also referred to herein as “DL-lactate”). Suitable pharmaceutically acceptable salts of DL-lactic acid include sodium DL-lactate, calcium DL-lactate and potassium DL-lactate. In one embodiment, the pharmaceutically acceptable salt of DL-lactic acid is sodium DL-lactate. In one embodiment, the concentration of DL-lactic acid or a pharmaceutically acceptable salt thereof is 200-1000 mM, 400-1000 mM, 200-800 mM, 400-800 mM, 200-600 mM, 400-600 mM, 200-400 mM, 200-300 mM, 300-500 mM, about 400 mM or about 200 mM.

It should be noted that the pharmaceutically acceptable salt of D-lactate or DL-lactate can be formed in situ by adding D-lactic acid or DL-lactic acid to the composition and then adjusting the pH (e.g. by addition of a base such as NaOH) to form D-lactate or DL-lactate. It is expected that D-lactate added to the composition in the form of sodium D-lactate would have the same effect as the addition of the same quantity (molar) of lactic acid, with a subsequent pH adjustment step using sodium hydroxide.

As described above, it has been discovered that the currently marketed VancoReady™ composition can be modified to replace the stabilizer NADA with D-lactate, while maintaining a composition with good stability. As shown in Examples 4-6, the present inventors have also discovered that further modification of the D-lactate composition to replace PEG400 with propylene glycol provides a surprising relative increase in stability. As shown in Example 9, this stabilizing effect was also shown for polyols sucrose, trehalose, glycerol and mannitol. As shown in Example 7, a combination of two polyols (propylene glycol and glycerol) also provided a stabilizing effect. Thus, compositions of the invention comprise a polyol selected from the group consisting of propylene glycol, sucrose, glycerol, trehalose, lactose, glucose, sorbitol and mannitol, or a mixture thereof.

In one embodiment, the concentration of polyol (or the combined total concentration of polyol if two or more polyols are present) in the composition is 200-1200 mM, such as 300-800 mM.

In one embodiment, the composition of the invention comprises a single polyol selected from the group consisting of propylene glycol, sucrose, glycerol, trehalose, lactose, glucose, sorbitol and mannitol. In another embodiment, the composition comprises a mixture of two polyols selected from the group consisting of propylene glycol, sucrose, glycerol, trehalose, lactose, glucose, sorbitol and mannitol. In one embodiment, the or each polyol is selected from the group consisting of sucrose, glycerol, trehalose, lactose, glucose, sorbitol and mannitol. In one embodiment, the or each polyol is selected from the group consisting of propylene glycol, sucrose, trehalose, lactose, glucose, sorbitol and mannitol. In one embodiment, the or each polyol is selected from the group consisting of sucrose, trehalose, lactose, glucose, sorbitol and mannitol.

In one embodiment, the single polyol is propylene glycol, which is suitably present in the composition at a concentration of 660-1200 mM, e.g. 700-1200 mM, 750-1200 mM, 800-1200 mM or 850-1200 mM. In one embodiment, the single polyol is glycerol, which is suitably present in the composition at a concentration of 200-1200 mM, such as 300-800 mM. In one embodiment, the single polyol is sucrose, which is suitably present in the composition at a concentration of 200-1200 mM, such as 300-800 mM. In one embodiment, the single polyol is trehalose, which is suitably present in the composition at a concentration of 200-1200 mM, such as 300-800 mM. In one embodiment, the single polyol is lactose, which is suitably present in the composition at a concentration of 200-1200 mM, such as 300-800 mM. In one embodiment, the single polyol is glucose, which is suitably present in the composition at a concentration of 200-1200 mM, such as 300-800 mM. In one embodiment, the single polyol is sorbitol, which is suitably present in the composition at a concentration of 200-1200 mM, such as 300-800 mM. In one embodiment, the single polyol is mannitol, which is suitably present in the composition at a concentration of 200-1200 mM, such as 300-800 mM.

In one embodiment, the composition comprises a mixture of propylene glycol and a polyol selected from the group consisting of sucrose, glycerol, trehalose, lactose, glucose, sorbitol and mannitol. In this embodiment, suitably the propylene glycol is present in the composition at a concentration of 10-160 mM, e.g. 10-150 mM or 10-125 mM, and the polyol selected from the group consisting of sucrose, glycerol, trehalose, lactose, glucose, sorbitol and mannitol is present in the composition at a concentration of 40-1190 mM, e.g. 50-1190 mM or 75-1190 mM. In this embodiment, the combined total concentration of propylene glycol and polyol selected from the group consisting of sucrose, glycerol, trehalose, lactose, glucose, sorbitol and mannitol is suitably 200-1200 mM, such as 300-800 mM.

In one embodiment, the polyol is a mixture of propylene glycol and glycerol. In this embodiment, suitably the propylene glycol is present in the composition at a concentration of 10-160 mM, e.g. 10-150 mM or 10-125 mM, and the glycerol is present in the composition at a concentration of 40-1190 mM, e.g. 50-1190 mM or 75-1190 mM. In this embodiment, the combined total concentration of propylene glycol and glycerol is suitably 200-1200 mM, such as 300-800 mM.

In one embodiment, the polyol is a mixture of propylene glycol and sucrose. In this embodiment, suitably the propylene glycol is present in the composition at a concentration of 10-160 mM, e.g. 10-150 mM or 10-125 mM, and the sucrose is present in the composition at a concentration of 40-1190 mM, e.g. 50-1190 mM or 75-1190 mM. In this embodiment, the combined total concentration of propylene glycol and sucrose is suitably 200-1200 mM, such as 300-800 mM.

In one embodiment, the polyol is a mixture of propylene glycol and mannitol. In this embodiment, suitably the propylene glycol is present in the composition at a concentration of 10-160 mM, e.g. 10-150 mM or 10-125 mM, and the mannitol is present in the composition at a concentration of 40-1190 mM, e.g. 50-1190 mM or 75-1190 mM. In this embodiment, the combined total concentration of propylene glycol and mannitol is suitably 200-1200 mM, such as 300-800 mM.

In one embodiment, the molar ratio of vancomycin or a pharmaceutically acceptable salt thereof, to polyol (or combined molar concentration if more than one polyol is present) is between 1:100 and 1:1000, e.g. between 1:100 and 1:500, in particular between 1:100 and 1:250.

The ready-to-administer compositions of the present invention can be administered directly to a patient without dilution. In order to minimise the risk of haemolysis and patient discomfort during administration hypotonic compositions (such as compositions with osmolarity less than 300 mOsm/L) and strongly hypertonic compositions should be avoided. It is generally accepted that compositions with osmolarity up to about 700 mOsm/L can be used for intravenous delivery without a significant risk of patient discomfort or haemolysis. For compositions where small uncharged species such as polyols represent the key ingredients, the maximum osmolarity limit can be higher. In any case, however, osmolarity above 1250 mOsm/L should be avoided for ready-to-administer compositions. Consequently, whilst higher concentrations of polyols have been shown to result in improved stability of vancomycin, their concentration in the compositions of the present invention is limited to ensure osmolarity does not exceed 1250 mOsm/L.

For the avoidance of doubt, any reference herein to “osmolarity” refers to a calculated osmolarity. Osmolarity is calculated as the sum of the molarities of all individual species dissolved in the solution and is expressed as osmoles per litre (Osm/L) or milliosmoles per litre (mOsm/L). For species that do not dissociate in the solution, the osmolarity (in Osm/L or mOsm/L) is equal to molarity (in moles/L or mmoles/L). For example, 500 mM glycerol in a solution at pH 5.0 does not dissociate, such that the solution has an osmolarity of 500 mOsm/L. For species that do dissociate in the solution, the osmolarity is the sum of the molarities of all dissociated species. For example, 500 mM sodium chloride at pH 5.0 dissociates into 500 mM sodium cation and 500 mM chloride anion, such that the solution has an osmolarity of 1000 mOsm/L.

The osmolarity of the composition of the invention is 400-1250 mOsm/L. In one embodiment, the osmolarity of the composition is 410-1250 mOsm/L, 425-1250 mOsm/L, 450-1250 mOsm/L, 500-1250 mOsm/L, 550-1250 mOsm/L, 600-1250 mOsm/L, 700-1250 mOsm/L, 400-1100 mOsm/L, 410-1100 mOsm/L, 425-1100 mOsm/L, 450-1100 mOsm/L, 500-1100 mOsm/L, 550-1100 mOsm/L, 600-1100 mOsm/L, 700-1100 mOsm/L, 400-1000 mOsm/L, 410-1000 mOsm/L, 425-1000 mOsm/L, 450-1000 mOsm/L, 500-1000 mOsm/L, 550-1000 mOsm/L, 600-1000 mOsm/L, 700-1000 mOsm/L, 400-800 mOsm/L, 410-800 mOsm/L, 425-800 mOsm/L, 450-800 mOsm/L, 500-800 mOsm/L, 550-800 mOsm/L, 600-800 mOsm/L, 700-800 mOsm/L, 400-600 mOsm/L, 410-600 mOsm/L, 425-600 mOsm/L, 450-600 mOsm/L, 500-600 mOsm/L or 500-600 mOsm/L.

Suitably, the composition is slightly hypertonic.

The composition of the invention is an aqueous solution composition and comprises at least 80% (v/v) water, such as at least 70% (v/v), at least 60% (v/v), at least 55% (v/v), at least 50% (v/v), at least 45% (v/v), at least 40% (v/v), at least 35% (v/v), at least 30% (v/v), at least 25% (v/v), at least 20% (v/v), at least 15% (v/v), at least 10% (v/v), at least 5% (v/v) or at least 2.5% (v/v) of water, e.g. sterile water for injection or bacteriostatic water for injection.

In certain embodiments, the addition of an inorganic salt containing a metal cation has a further stabilizing effect on the compositions described herein, as shown in Examples 5-8.

Thus, in one embodiment, the composition further comprises an inorganic salt containing a metal cation, especially a Group 1 or Group 2 metal cation. In one embodiment, the inorganic salt containing a metal cation is selected from the group consisting of sodium chloride, calcium chloride, sodium oxamate and sodium sulphate. In one embodiment, the composition comprises an inorganic salt containing a metal cation, which is sodium chloride or calcium chloride, in particular sodium chloride. In one embodiment, the composition comprises an inorganic salt containing a metal cation, which is sodium oxamate. The concentration of inorganic salt containing a metal cation is typically 1-500 mM, e.g. 50-250 mM or 75-150 mM. The aforementioned concentrations of inorganic salt will typically represent the concentration of inorganic salt such as sodium chloride added to the composition.

The effect of pH on the stability of vancomycin compositions is shown in Example 2, where a range of pH values between 3.5 and 9.5 were tested, with the optimal pH being observed to be between 4.5 and 5.5. Thus, in one embodiment, the pH of the composition of the invention is in the range 4.0 to 6.0, such as about 5.0.

It should be noted that all references herein to “pH” refer to the pH of a composition evaluated at 21° C. All references to “pKₐ” refer to the pKₐ of an ionisable group evaluated at 25° C. (see CRC Handbook of Chemistry and Physics, 79th Edition, 1998, D. R. Lide).

In one embodiment, the composition further comprises an antioxidant, which is suitably selected from the group consisting of monothioglycerol, butylated hydroxyanisole, glutathione (reduced), ascorbate, cysteine and methionine, and is suitably methionine. When included, the antioxidant is typically present in the composition at a concentration of 1-100 mM, e.g. 10-50 mM or 25-50 mM.

In one embodiment, the composition of the invention is substantially free of, or free of amino acids. In one embodiment, the composition of the invention is substantially free of, or free of polyethylene glycol (PEG), in particular PEG400. In one embodiment, the composition of the invention is substantially free of, or free of propylene glycol. In one embodiment, the composition of the invention is substantially free of, or free of glycerol. In one embodiment, the composition of the invention is substantially free of, or free of N-acetyl-D-alanine. In one embodiment, the composition of the invention is substantially free of, or free of ethanol. In one embodiment, the composition of the invention is substantially free of, or free of EDTA. In one embodiment, the composition of the invention is substantially free of, or free of cyclodextrins, e.g. sulfobutylether-betacycoldextrin. In all of these embodiments, “substantially free of” means that the composition contains less than 0.1 mM of the stated component.

The composition may comprise a non-ionic surfactant, which is suitably selected from the group consisting of an alkyl glycoside, a polysorbate, an alkyl ether of polyethylene glycol, a block copolymer of polyethylene glycol and polypropylene glycol, and an alkylphenyl ether of polyethylene glycol.

When the non-ionic surfactant is an alkyl glycoside, it is suitably selected from the group consisting of dodecyl maltoside, dodecyl glucoside, octyl glucoside, octyl maltoside, decyl glucoside, decyl maltoside, decyl glucopyranoside, tridecyl glucoside, tridecyl maltoside, tetradecyl glucoside, tetradecyl maltoside, hexadecyl glucoside, hexadecyl maltoside, sucrose monooctanoate, sucrose monodecanoate, sucrose monododecanoate, sucrose monotridecanoate, sucrose monotetradecanoate and sucrose monohexadecanoate. In one embodiment, the alkyl glycoside is dodecyl maltoside or decyl glucopyranoside, and in particular is dodecyl maltoside.

When the non-ionic surfactant is a polysorbate, it is suitably selected from the group consisting of polysorbate 20, polysorbate 40, polysorbate 60 and polysorbate 80. In one embodiment, the non-ionic surfactant is polysorbate 20 or polysorbate 80. Polysorbates are known under a range of brand names including in particular Tween “XX”, and also Alkest TW “XX”, where “XX” is 20, 40, 60 or 80.

When the non-ionic surfactant is an alkyl ether of polyethylene glycol, it is suitably selected from the group consisting of polyethylene glycol (2) hexadecyl ether (Brij 52), polyethylene glycol (2) oleyl ether (Brij 93), polyethylene glycol (2) dodecyl ether (Brij L4), polyethylene glycol (4) lauryl ether (Brij 30), polyethylene glycol (10) lauryl ether (Brij 35), polyethylene glycol (20) hexadecyl ether (Brij 58) and polyethylene glycol (10) stearyl ether (Brij 78).

When the non-ionic surfactant is a block copolymer of polyethylene glycol and polypropylene glycol, it is suitably selected from the group consisting of poloxamer 188, poloxamer 407, poloxamer 171 or poloxamer 185. Poloxamers are also known under brand names Pluronics or Koliphors. For example, poloxamer 188 is marketed as Pluronic F-68.

When the non-ionic surfactant is an alkylphenyl ether of polyethylene glycol, it is suitably 4-(1,1,3,3-tetramethylbutyl)phenyl-polyethylene glycol, also known under a brand name Triton X-100.

When included, the concentration of non-ionic surfactant is typically in the range 1-5000 μg/mL, 1-1000 μg/mL, such as 5-500 μg/mL, 10-400 μg/mL, 20-400 μg/mL, 50-400 μg/mL, 10-300 μg/mL, 20-300 μg/mL, 50-300 μg/mL, 10-200 μg/mL, 20-200 μg/mL, 50-200 μg/mL, 10-100 g/mL, 20-100 μg/mL, 50-100 μg/mL or around 50 μg/mL.

The composition of the invention may additionally comprise a preservative such as a phenolic or benzylic preservative. The preservative is suitably selected from the group consisting of phenol, m-cresol, chlorocresol, benzyl alcohol, propyl paraben and methyl paraben, in particular phenol, m-cresol and benzyl alcohol, and mixtures thereof. When included, the concentration of preservative (total concentration if more than one preservative is present) is typically 10-100 mM, for example 20-80 mM, such as 25-50 mM. The optimal concentration of the preservative in the composition is selected to ensure the composition passes the Pharmacopoeia Antimicrobial Effectiveness Test (USP <51>, Vol. 32).

In one embodiment, there is provided a storage stable aqueous solution composition comprising:
vancomycin or a pharmaceutically acceptable salt thereof, at a concentration of 1-10 mg/mL, e.g. 2.5-7.5 mg/ml or about 5 mg/mL;
D-lactic acid or a pharmaceutically acceptable salt thereof, at a concentration of 100-500 mM, 100-300 mM, 100-200 mM, 100-150 mM, about 100 mM or about 200 mM; or DL-lactic acid or a pharmaceutically acceptable salt thereof, at a concentration of 200-1000 mM, 200-600 mM, 200-400 mM, 200-300 mM, about 200 mM or about 400 mM;
a polyol selected from the group consisting of propylene glycol, sucrose, glycerol, trehalose, lactose, glucose, sorbitol and mannitol, or a mixture thereof, at a total concentration of 200-1200 mM, such as 300-800 mM; and
optionally an inorganic salt containing a metal cation;
   wherein the osmolarity of the composition is 400-1250 mOsm/L, e.g. 450-1250 mOsm/L, or 550-1250 mOsm/L; and
   wherein the pH of the composition is in the range 4.0 to 6.0, such as about 5.0.

In one embodiment, there is provided a storage stable aqueous solution composition comprising:
vancomycin or a pharmaceutically acceptable salt thereof, at a concentration of 1-10 mg/mL, e.g. 2.5-7.5 mg/ml or about 5 mg/ml;
D-lactic acid or a pharmaceutically acceptable salt thereof, at a concentration of 100-500 mM, 100-300 mM, 100-200 mM, 100-150 mM, about 100 mM or about 200 mM; or DL-lactic acid or a pharmaceutically acceptable salt thereof, at a concentration of 200-1000 mM, 200-600 mM, 200-400 mM, 200-300 mM, about 200 mM or about 400 mM;
propylene glycol at a concentration of 660-1200 mM, e.g. 700-1200 mM, 750-1200 mM, 800-1200 mM or 850-1200 mM; and
optionally an inorganic salt containing a metal cation;
   wherein the osmolarity of the composition is 400-1250 mOsm/L, e.g. 450-1250 mOsm/L, or 550-1250 mOsm/L; and
   wherein the pH of the composition is in the range 4.0 to 6.0, such as about 5.0.

In one embodiment, there is provided a storage stable aqueous solution composition comprising:
vancomycin or a pharmaceutically acceptable salt thereof, at a concentration of 1-10 mg/mL, e.g. 2.5-7.5 mg/ml or about 5 mg/ml;
D-lactic acid or a pharmaceutically acceptable salt thereof, at a concentration of 100-500 mM, 100-300 mM, 100-200 mM, 100-150 mM, about 100 mM or about 200 mM; or DL-lactic acid or a pharmaceutically acceptable salt thereof, at a concentration of 200-1000 mM, 200-600 mM, 200-400 mM, 200-300 mM, about 200 mM or about 400 mM;
sucrose at a concentration of 200-1200 mM, such as 300-800 mM; and
optionally an inorganic salt containing a metal cation;
   wherein the osmolarity of the composition is 400-1250 mOsm/L, e.g. 450-1250 mOsm/L, or 550-1250 mOsm/L; and
   wherein the pH of the composition is in the range 4.0 to 6.0, such as about 5.0.

In one embodiment, there is provided a storage stable aqueous solution composition comprising:
vancomycin or a pharmaceutically acceptable salt thereof, at a concentration of 1-10 mg/mL, e.g. 2.5-7.5 mg/mL or about 5 mg/mL;
D-lactic acid or a pharmaceutically acceptable salt thereof, at a concentration of 100-500 mM, 100-300 mM, 100-200 mM, 100-150 mM, about 100 mM or about 200 mM; or DL-lactic acid or a pharmaceutically acceptable salt thereof, at a concentration of 200-1000 mM, 200-600 mM, 200-400 mM, 200-300 mM, about 200 mM or about 400 mM;
a mixture of two polyols selected from the group consisting of propylene glycol, sucrose, glycerol, trehalose, lactose, glucose, sorbitol and mannitol, at a combined total concentration of 200-1200 mM, such as 300-800 mM; and
optionally an inorganic salt containing a metal cation;
   wherein the osmolarity of the composition is 400-1250 mOsm/L, e.g. 450-1250 mOsm/L, or 550-1250 mOsm/L; and
   wherein the pH of the composition is in the range 4.0 to 6.0, such as about 5.0.

In one embodiment, there is provided a storage stable aqueous solution composition comprising:
vancomycin or a pharmaceutically acceptable salt thereof, at a concentration of 1-10 mg/mL, e.g. 2.5-7.5 mg/mL or about 5 mg/mL;
D-lactic acid or a pharmaceutically acceptable salt thereof, at a concentration of 100-500 mM, 100-300 mM, 100-200 mM, 100-150 mM, about 100 mM or about 200 mM; or DL-lactic acid or a pharmaceutically acceptable salt thereof, at a concentration of 200-1000 mM, 200-600 mM, 200-400 mM, 200-300 mM, about 200 mM or about 400 mM;
a mixture of propylene glycol at a concentration of 10-160 mM, e.g. 10-150 mM or 10-125 mM, and a polyol selected from the group consisting of sucrose, glycerol, trehalose, lactose, glucose, sorbitol and mannitol, at a concentration of 40-1190 mM, e.g. 50-1190 mM or 75-1190 mM; wherein the combined total concentration of propylene glycol and polyol selected from the group consisting of sucrose, glycerol, trehalose, lactose, glucose, sorbitol and mannitol is 200-1200 mM, such as 300-800 mM; and
optionally an inorganic salt containing a metal cation;
   wherein the osmolarity of the composition is 400-1250 mOsm/L, e.g. 450-1250 mOsm/L, or 550-1250 mOsm/L; and
   wherein the pH of the composition is in the range 4.0 to 6.0, such as about 5.0.

In one embodiment, there is provided a storage stable aqueous solution composition comprising:
vancomycin or a pharmaceutically acceptable salt thereof, at a concentration of 1-10 mg/mL, e.g. 2.5-7.5 mg/mL or about 5 mg/ml;
D-lactic acid or a pharmaceutically acceptable salt thereof, at a concentration of 100-500 mM, 100-300 mM, 100-200 mM, 100-150 mM, about 100 mM or about 200 mM;
a polyol selected from the group consisting of propylene glycol, sucrose, glycerol, trehalose, lactose, glucose, sorbitol and mannitol, or a mixture thereof, at a total concentration of 200-1200 mM, such as 300-800 mM; and
optionally an inorganic salt containing a metal cation;
   wherein the osmolarity of the composition is 400-1250 mOsm/L, e.g. 450-1250 mOsm/L, or 550-1250 mOsm/L; and
   wherein the pH of the composition is in the range 4.0 to 6.0, such as about 5.0.

In one embodiment, there is provided a storage stable aqueous solution composition comprising:
vancomycin or a pharmaceutically acceptable salt thereof, at a concentration of 1-10 mg/mL, e.g. 2.5-7.5 mg/ml or about 5 mg/mL;
D-lactic acid or a pharmaceutically acceptable salt thereof, at a concentration of 100-500 mM, 100-300 mM, 100-200 mM, 100-150 mM, about 100 mM or about 200 mM;
propylene glycol at a concentration of 660-1200 mM, e.g. 700-1200 mM, 750-1200 mM, 800-1200 mM or 850-1200 mM; and
optionally an inorganic salt containing a metal cation;
   wherein the osmolarity of the composition is 400-1250 mOsm/L, e.g. 450-1250 mOsm/L, or 550-1250 mOsm/L; and
   wherein the pH of the composition is in the range 4.0 to 6.0, such as about 5.0.

In one embodiment, there is provided a storage stable aqueous solution composition comprising:
vancomycin or a pharmaceutically acceptable salt thereof, at a concentration of 1-10 mg/mL, e.g. 2.5-7.5 mg/mL or about 5 mg/mL;
D-lactic acid or a pharmaceutically acceptable salt thereof, at a concentration of 100-500 mM, 100-300 mM, 100-200 mM, 100-150 mM, about 100 mM or about 200 mM;
propylene glycol at a concentration of 660-1200 mM, e.g. 700-1200 mM, 750-1200 mM, 800-1200 mM or 850-1200 mM; and
an inorganic salt containing a metal cation at a concentration of 1-500 mM e.g. 50-250 mM;
   wherein the osmolarity of the composition is 400-1250 mOsm/L, e.g. 450-1250 mOsm/L, or 550-1250 mOsm/L; and
   wherein the pH of the composition is in the range 4.0 to 6.0, such as about 5.0.

In one embodiment, there is provided a storage stable aqueous solution composition comprising:
vancomycin or a pharmaceutically acceptable salt thereof, e.g. vancomycin hydrochloride, at a concentration of 1-10 mg/mL, e.g. 2.5-7.5 mg/ml or about 5 mg/mL;
D-lactic acid or a pharmaceutically acceptable salt thereof, e.g. sodium D-lactate, at a concentration of 100-500 mM, 100-300 mM, 100-200 mM, 100-150 mM, about 100 mM or about 200 mM;
propylene glycol at a concentration of 660-1200 mM, e.g. 700-1200 mM, 750-1200 mM, 800-1200 mM or 850-1200 mM; and
sodium chloride at a concentration of 1-500 mM, e.g. 50-250 mM;
   wherein the osmolarity of the composition is 400-1250 mOsm/L, e.g. 450-1250 mOsm/L, or 550-1250 mOsm/L; and
   wherein the pH of the composition is in the range 4.0 to 6.0, such as about 5.0.

In one embodiment, there is provided a storage stable aqueous solution composition comprising:
vancomycin or a pharmaceutically acceptable salt thereof, e.g. vancomycin hydrochloride, at a concentration of about 5 mg/mL;
D-lactic acid or a pharmaceutically acceptable salt thereof, e.g. sodium D-lactate, at a concentration of 100-200 mM;
propylene glycol at a concentration of 660-1200 mM; and
sodium chloride at a concentration of 50-250 mM;
   wherein the osmolarity of the composition is 550-1250 mOsm/L; and
   wherein the pH of the composition is in the range 4.0 to 6.0.

In one embodiment, there is provided a storage stable aqueous solution composition comprising:
vancomycin or a pharmaceutically acceptable salt thereof, at a concentration of 1-10 mg/mL, e.g. 2.5-7.5 mg/ml or about 5 mg/ml;
D-lactic acid or a pharmaceutically acceptable salt thereof, at a concentration of 100-500 mM, 100-300 mM, 100-200 mM, 100-150 mM, about 100 mM or about 200 mM;
glycerol at a concentration of 200-1200 mM, such as 300-800 mM; and
optionally an inorganic salt containing a metal cation;
   wherein the osmolarity of the composition is 400-1250 mOsm/L, e.g. 400-1000 mOsm/L, or 500-800 mOsm/L; and
   wherein the pH of the composition is in the range 4.0 to 6.0, such as about 5.0.

In one embodiment, there is provided a storage stable aqueous solution composition comprising:
vancomycin or a pharmaceutically acceptable salt thereof, e.g. vancomycin hydrochloride, at a concentration of 1-10 mg/mL, e.g. 2.5-7.5 mg/ml or about 5 mg/mL;
D-lactic acid or a pharmaceutically acceptable salt thereof, e.g. sodium D-lactate, at a concentration of 100-500 mM, 100-300 mM, 100-200 mM, 100-150 mM, about 100 mM or about 200 mM; and
glycerol at a concentration of 200-1200 mM, such as 300-800 mM;
   wherein the osmolarity of the composition is 400-1250 mOsm/L, e.g. 400-1000 mOsm/L, or 500-800 mOsm/L; and
   wherein the pH of the composition is in the range 4.0 to 6.0, such as about 5.0.

In one embodiment, there is provided a storage stable aqueous solution composition comprising:
vancomycin or a pharmaceutically acceptable salt thereof, e.g. vancomycin hydrochloride, at a concentration of about 5 mg/ml;
D-lactic acid or a pharmaceutically acceptable salt thereof, e.g. sodium D-lactate, at a concentration of 100-200 mM, 100-150 mM e.g. about 100 mM or about 200 mM; and
glycerol at a concentration of 300-800 mM;
   wherein the osmolarity of the composition is 500-800 mOsm/L; and
   wherein the pH of the composition is in the range 4.0 to 6.0.

In one embodiment, there is provided a storage stable aqueous solution composition comprising:
vancomycin or a pharmaceutically acceptable salt thereof, at a concentration of 1-10 mg/mL, e.g. 2.5-7.5 mg/mL or about 5 mg/mL;
D-lactic acid or a pharmaceutically acceptable salt thereof, at a concentration of 100-500 mM, 100-300 mM, 100-200 mM, 100-150 mM, about 100 mM or about 200 mM;
sucrose at a concentration of 200-1200 mM, such as 300-800 mM; and
optionally an inorganic salt containing a metal cation;
   wherein the osmolarity of the composition is 400-1250 mOsm/L, e.g. 450-1250 mOsm/L, or 550-1250 mOsm/L; and
   wherein the pH of the composition is in the range 4.0 to 6.0, such as about 5.0.

In one embodiment, there is provided a storage stable aqueous solution composition comprising:
vancomycin or a pharmaceutically acceptable salt thereof, at a concentration of 1-10 mg/mL, e.g. 2.5-7.5 mg/ml or about 5 mg/ml;
D-lactic acid or a pharmaceutically acceptable salt thereof, at a concentration of 100-500 mM, 100-300 mM, 100-200 mM, 100-150 mM, about 100 mM or about 200 mM;
a mixture of two polyols selected from the group consisting of propylene glycol, sucrose, glycerol, trehalose, lactose, glucose, sorbitol and mannitol, at a combined total concentration of 200-1200 mM, such as 300-800 mM; and
optionally an inorganic salt containing a metal cation;
   wherein the osmolarity of the composition is 400-1250 mOsm/L, e.g. 450-1250 mOsm/L, or 550-1250 mOsm/L; and
   wherein the pH of the composition is in the range 4.0 to 6.0, such as about 5.0.

In one embodiment, there is provided a storage stable aqueous solution composition comprising:
vancomycin or a pharmaceutically acceptable salt thereof, at a concentration of 1-10 mg/mL, e.g. 2.5-7.5 mg/mL or about 5 mg/mL;
D-lactic acid or a pharmaceutically acceptable salt thereof, at a concentration of 100-500 mM, 100-300 mM, 100-200 mM, 100-150 mM, about 100 mM or about 200 mM;
a mixture of propylene glycol at a concentration of 10-160 mM, e.g. 10-150 mM or 10-125 mM, and a polyol selected from the group consisting of sucrose, glycerol, trehalose, lactose, glucose, sorbitol and mannitol, at a concentration of 40-1190 mM, e.g. 50-1190 mM or 75-1190 mM; wherein the combined total concentration of propylene glycol and polyol selected from the group consisting of sucrose, glycerol, trehalose, lactose, glucose, sorbitol and mannitol is 200-1200 mM, such as 300-800 mM; and
optionally an inorganic salt containing a metal cation;
   wherein the osmolarity of the composition is 400-1250 mOsm/L, e.g. 450-1250 mOsm/L, or 550-1250 mOsm/L; and
   wherein the pH of the composition is in the range 4.0 to 6.0, such as about 5.0.

In one embodiment, there is provided a storage stable aqueous solution composition comprising:
vancomycin or a pharmaceutically acceptable salt thereof, at a concentration of 1-10 mg/mL, e.g. 2.5-7.5 mg/mL or about 5 mg/ml;
DL-lactic acid or a pharmaceutically acceptable salt thereof, at a concentration of 200-1000 mM, 200-600 mM, 200-400 mM, 200-300 mM, about 200 mM or about 400 mM;
a polyol selected from the group consisting of propylene glycol, sucrose, glycerol, trehalose, lactose, glucose, sorbitol and mannitol, or a mixture thereof, at a total concentration of 200-1200 mM, such as 300-800 mM; and
optionally an inorganic salt containing a metal cation;
   wherein the osmolarity of the composition is 400-1250 mOsm/L, e.g. 450-1250 mOsm/L, or 550-1250 mOsm/L; and
   wherein the pH of the composition is in the range 4.0 to 6.0, such as about 5.0.

In one embodiment, there is provided a storage stable aqueous solution composition comprising:
vancomycin or a pharmaceutically acceptable salt thereof, at a concentration of 1-10 mg/mL, e.g. 2.5-7.5 mg/ml or about 5 mg/ml;
DL-lactic acid or a pharmaceutically acceptable salt thereof, at a concentration of 200-1000 mM, 200-600 mM, 200-400 mM, 200-300 mM, about 200 mM or about 400 mM;
propylene glycol at a concentration of 660-1200 mM, e.g. 700-1200 mM, 750-1200 mM, 800-1200 mM or 850-1200 mM; and
optionally an inorganic salt containing a metal cation;
   wherein the osmolarity of the composition is 400-1250 mOsm/L, e.g. 450-1250 mOsm/L, or 550-1250 mOsm/L; and
   wherein the pH of the composition is in the range 4.0 to 6.0, such as about 5.0.

In one embodiment, there is provided a storage stable aqueous solution composition comprising:
vancomycin or a pharmaceutically acceptable salt thereof, at a concentration of 1-10 mg/mL, e.g. 2.5-7.5 mg/ml or about 5 mg/mL;
DL-lactic acid or a pharmaceutically acceptable salt thereof, at a concentration of 200-1000 mM, 200-600 mM, 200-400 mM, 200-300 mM, about 200 mM or about 400 mM;
glycerol at a concentration of 200-1200 mM, such as 300-800 mM; and
optionally an inorganic salt containing a metal cation;
   wherein the osmolarity of the composition is 400-1250 mOsm/L, e.g. 400-1000 mOsm/L, or 500-800 mOsm/L; and
   wherein the pH of the composition is in the range 4.0 to 6.0, such as about 5.0.

In one embodiment, there is provided a storage stable aqueous solution composition comprising:
vancomycin or a pharmaceutically acceptable salt thereof, e.g. vancomycin hydrochloride, at a concentration of 1-10 mg/mL, e.g. 2.5-7.5 mg/ml or about 5 mg/mL;
DL-lactic acid or a pharmaceutically acceptable salt thereof, e.g. sodium DL-lactate, at a concentration of 200-1000 mM, 200-600 mM, 200-400 mM, 200-300 mM, about 200 mM or about 400 mM; and
glycerol at a total concentration of 200-1200 mM, such as 300-800 mM;
   wherein the osmolarity of the composition is 400-1250 mOsm/L, e.g. 400-1000 mOsm/L, or 500-800 mOsm/L; and
   wherein the pH of the composition is in the range 4.0 to 6.0, such as about 5.0.

In one embodiment, there is provided a storage stable aqueous solution composition comprising:
vancomycin or a pharmaceutically acceptable salt thereof, e.g. vancomycin hydrochloride, at a concentration of about 5 mg/ml;
DL-lactic acid or a pharmaceutically acceptable salt thereof, e.g. sodium DL-lactate, at a concentration of 200-400 mM, 200-300 mM, e.g. about 200 mM or about 400 mM; and
glycerol at a concentration of 300-800 mM;
   wherein the osmolarity of the composition is 500-800 mOsm/L; and
   wherein the pH of the composition is in the range 4.0 to 6.0.

In one embodiment, there is provided a storage stable aqueous solution composition comprising:
vancomycin or a pharmaceutically acceptable salt thereof, at a concentration of 1-10 mg/mL, e.g. 2.5-7.5 mg/ml or about 5 mg/mL;
DL-lactic acid or a pharmaceutically acceptable salt thereof, at a concentration of 200-1000 mM, 200-600 mM, 200-400 mM, 200-300 mM, about 200 mM or about 400 mM;
sucrose at a concentration of 200-1200 mM, such as 300-800 mM; and
optionally an inorganic salt containing a metal cation;
   wherein the osmolarity of the composition is 400-1250 mOsm/L, e.g. 450-1250 mOsm/L, or 550-1250 mOsm/L; and
   wherein the pH of the composition is in the range 4.0 to 6.0, such as about 5.0.

In one embodiment, there is provided a storage stable aqueous solution composition comprising:
vancomycin or a pharmaceutically acceptable salt thereof, at a concentration of 1-10 mg/mL, e.g. 2.5-7.5 mg/ml or about 5 mg/mL;
DL-lactic acid or a pharmaceutically acceptable salt thereof, at a concentration of 200-1000 mM, 200-600 mM, 200-400 mM, 200-300 mM, about 200 mM or about 400 mM;
a mixture of two polyols selected from the group consisting of propylene glycol, sucrose, glycerol, trehalose, lactose, glucose, sorbitol and mannitol, at a combined total concentration of 200-1200 mM, such as 300-800 mM; and
optionally an inorganic salt containing a metal cation;
   wherein the osmolarity of the composition is 400-1250 mOsm/L, e.g. 450-1250 mOsm/L, or 550-1250 mOsm/L; and
   wherein the pH of the composition is in the range 4.0 to 6.0, such as about 5.0.

In one embodiment, there is provided a storage stable aqueous solution composition comprising:
vancomycin or a pharmaceutically acceptable salt thereof, at a concentration of 1-10 mg/mL, e.g. 2.5-7.5 mg/mL or about 5 mg/ml;
DL-lactic acid or a pharmaceutically acceptable salt thereof, at a concentration of 200-1000 mM, 200-600 mM, 200-400 mM, 200-300 mM, about 200 mM or about 400 mM;
a mixture of propylene glycol at a concentration of 10-160 mM, e.g. 10-150 mM or 10-125 mM, and a polyol selected from the group consisting of sucrose, glycerol, trehalose, lactose, glucose, sorbitol and mannitol, at a concentration of 40-1190 mM, e.g. 50-1190 mM or 75-1190 mM; wherein the combined total concentration of propylene glycol and polyol selected from the group consisting of sucrose, glycerol, trehalose, lactose, glucose, sorbitol and mannitol is 200-1200 mM, such as 300-800 mM; and
optionally an inorganic salt containing a metal cation;
   wherein the osmolarity of the composition is 400-1250 mOsm/L, e.g. 450-1250 mOsm/L, or 550-1250 mOsm/L; and
   wherein the pH of the composition is in the range 4.0 to 6.0, such as about 5.0.

In one embodiment, there is provided a storage stable aqueous solution composition consisting of, or consisting essentially of:
(i) vancomycin or a pharmaceutically acceptable salt thereof, at a concentration of 1-10 mg/mL, e.g. 2.5-7.5 mg/mL or about 5 mg/mL;
(ii) D-lactic acid or a pharmaceutically acceptable salt thereof, at a concentration of 100-500 mM, 100-300 mM, 100-200 mM, 100-150 mM, about 100 mM or about 200 mM; or DL-lactic acid or a pharmaceutically acceptable salt thereof, at a concentration of 200-1000 mM, 200-600 mM, 200-400 mM, 200-300 mM, about 200 mM or about 400 mM;
(iii) a polyol selected from the group consisting of propylene glycol, sucrose, glycerol, trehalose, lactose, glucose, sorbitol and mannitol, or a mixture thereof, at a total concentration of 200-1200 mM, such as 300-800 mM;
(iv) water, e.g. water for injection;
(v) optionally an inorganic salt containing a metal cation;
(vi) optionally an antioxidant;
(vii) optionally a non-ionic surfactant; and
(viii) optionally a preservative;
   wherein the osmolarity of the composition is 400-1250 mOsm/L, e.g. 450-1250 mOsm/L, or 550-1250 mOsm/L; and
   wherein the pH of the composition is in the range 4.0 to 6.0, such as about 5.0.

In one embodiment, there is provided a storage stable aqueous solution composition consisting of, or consisting essentially of:
(i) vancomycin or a pharmaceutically acceptable salt thereof, at a concentration of 1-10 mg/mL, e.g. 2.5-7.5 mg/ml or about 5 mg/ml;
(ii) D-lactic acid or a pharmaceutically acceptable salt thereof, at a concentration of 100-500 mM, 100-300 mM, 100-200 mM, 100-150 mM, about 100 mM or about 200 mM; or DL-lactic acid or a pharmaceutically acceptable salt thereof, at a concentration of 200-1000 mM, 200-600 mM, 200-400 mM, 200-300 mM, about 200 mM or about 400 mM;
(iii) propylene glycol at a concentration of 660-1200 mM, e.g. 700-1200 mM, 750-1200 mM, 800-1200 mM or 850-1200 mM;
(iv) water, e.g. water for injection;
(v) optionally an inorganic salt containing a metal cation;
(vi) optionally an antioxidant;
(vii) optionally a non-ionic surfactant; and
(viii) optionally a preservative;
   wherein the osmolarity of the composition is 400-1250 mOsm/L, e.g. 450-1250 mOsm/L, or 550-1250 mOsm/L; and
   wherein the pH of the composition is in the range 4.0 to 6.0, such as about 5.0.

In one embodiment, there is provided a storage stable aqueous solution composition consisting of, or consisting essentially of:
(i) vancomycin or a pharmaceutically acceptable salt thereof, at a concentration of 1-10 mg/mL, e.g. 2.5-7.5 mg/ml or about 5 mg/mL;
(ii) D-lactic acid or a pharmaceutically acceptable salt thereof, at a concentration of 100-500 mM, 100-300 mM, 100-200 mM, 100-150 mM, about 100 mM or about 200 mM; or DL-lactic acid or a pharmaceutically acceptable salt thereof, at a concentration of 200-1000 mM, 200-600 mM, 200-400 mM, 200-300 mM, about 200 mM or about 400 mM;
(iii) sucrose at a concentration of 200-1200 mM, such as 300-800 mM;
(iv) water, e.g. water for injection;
(v) optionally an inorganic salt containing a metal cation;
(vi) optionally an antioxidant;
(vii) optionally a non-ionic surfactant; and
(viii) optionally a preservative;
   wherein the osmolarity of the composition is 400-1250 mOsm/L, e.g. 450-1250 mOsm/L, or 550-1250 mOsm/L; and
   wherein the pH of the composition is in the range 4.0 to 6.0, such as about 5.0.

In one embodiment, there is provided a storage stable aqueous solution composition consisting of, or consisting essentially of:
(i) vancomycin or a pharmaceutically acceptable salt thereof, at a concentration of 1-10 mg/mL, e.g. 2.5-7.5 mg/mL or about 5 mg/ml;
(ii) D-lactic acid or a pharmaceutically acceptable salt thereof, at a concentration of 100-500 mM, 100-300 mM, 100-200 mM, 100-150 mM, about 100 mM or about 200 mM; or DL-lactic acid or a pharmaceutically acceptable salt thereof, at a concentration of 200-1000 mM, 200-600 mM, 200-400 mM, 200-300 mM, about 200 mM or about 400 mM;
(iii) a mixture of two polyols selected from the group consisting of propylene glycol, sucrose, glycerol, trehalose, lactose, glucose, sorbitol and mannitol, at a combined total concentration of 200-1200 mM, such as 300-800 mM;
(iv) water, e.g. water for injection;
(v) optionally an inorganic salt containing a metal cation;
(vi) optionally an antioxidant;
(vii) optionally a non-ionic surfactant; and
(viii) optionally a preservative;
   wherein the osmolarity of the composition is 400-1250 mOsm/L, e.g. 450-1250 mOsm/L, or 550-1250 mOsm/L; and
   wherein the pH of the composition is in the range 4.0 to 6.0, such as about 5.0.

In one embodiment, there is provided a storage stable aqueous solution composition consisting of, or consisting essentially of:
(i) vancomycin or a pharmaceutically acceptable salt thereof, at a concentration of 1-10 mg/mL, e.g. 2.5-7.5 mg/ml or about 5 mg/mL;
(ii) D-lactic acid or a pharmaceutically acceptable salt thereof, at a concentration of 100-500 mM, 100-300 mM, 100-200 mM, 100-150 mM, about 100 mM or about 200 mM; or DL-lactic acid or a pharmaceutically acceptable salt thereof, at a concentration of 200-1000 mM, 200-600 mM, 200-400 mM, 200-300 mM, about 200 mM or about 400 mM;
(iii) a mixture of propylene glycol at a concentration of 10-160 mM, e.g. 10-150 mM or 10-125 mM, and a polyol selected from the group consisting of sucrose, glycerol, trehalose, lactose, glucose, sorbitol and mannitol, at a concentration of 40-1190 mM, e.g. 50-1190 mM or 75-1190 mM; wherein the combined total concentration of propylene glycol and polyol selected from the group consisting of sucrose, glycerol, trehalose, lactose, glucose, sorbitol and mannitol is 200-1200 mM, such as 300-800 mM;
(iv) water, e.g. water for injection;
(v) optionally an inorganic salt containing a metal cation;
(vi) optionally an antioxidant;
(vii) optionally a non-ionic surfactant; and
(viii) optionally a preservative;
   wherein the osmolarity of the composition is 400-1250 mOsm/L, e.g. 450-1250 mOsm/L, or 550-1250 mOsm/L; and
   wherein the pH of the composition is in the range 4.0 to 6.0, such as about 5.0.

In one embodiment, there is provided a storage stable aqueous solution composition consisting of, or consisting essentially of:
(i) vancomycin or a pharmaceutically acceptable salt thereof, at a concentration of 1-10 mg/mL, e.g. 2.5-7.5 mg/ml or about 5 mg/ml;
(ii) D-lactic acid or a pharmaceutically acceptable salt thereof, at a concentration of 100-500 mM, 100-300 mM, 100-200 mM, 100-150 mM, about 100 mM or about 200 mM;
(iii) a polyol selected from the group consisting of propylene glycol, sucrose, glycerol, trehalose, lactose, glucose, sorbitol and mannitol, or a mixture thereof, at a total concentration of 200-1200 mM, such as 300-800 mM;
(iv) water, e.g. water for injection;
(v) optionally an inorganic salt containing a metal cation;
(vi) optionally an antioxidant;
(vii) optionally a non-ionic surfactant; and
(viii) optionally a preservative;
   wherein the osmolarity of the composition is 400-1250 mOsm/L, e.g. 450-1250 mOsm/L, or 550-1250 mOsm/L; and
   wherein the pH of the composition is in the range 4.0 to 6.0, such as about 5.0.

In one embodiment, there is provided a storage stable aqueous solution composition consisting of, or consisting essentially of:
(i) vancomycin or a pharmaceutically acceptable salt thereof, at a concentration of 1-10 mg/mL, e.g. 2.5-7.5 mg/ml or about 5 mg/ml;
(ii) D-lactic acid or a pharmaceutically acceptable salt thereof, at a concentration of 100-500 mM, 100-300 mM, 100-200 mM, 100-150 mM, about 100 mM or about 200 mM;
(iii) propylene glycol at a concentration of 660-1200 mM, e.g. 700-1200 mM, 750-1200 mM, 800-1200 mM or 850-1200 mM;
(iv) water, e.g. water for injection;
(v) optionally an inorganic salt containing a metal cation;
(vi) optionally an antioxidant;
(vii) optionally a non-ionic surfactant; and
(viii) optionally a preservative;
   wherein the osmolarity of the composition is 400-1250 mOsm/L, e.g. 450-1250 mOsm/L, or 550-1250 mOsm/L; and
   wherein the pH of the composition is in the range 4.0 to 6.0, such as about 5.0.

In one embodiment, there is provided a storage stable aqueous solution composition consisting of, or consisting essentially of:
(i) vancomycin or a pharmaceutically acceptable salt thereof, at a concentration of 1-10 mg/mL, e.g. 2.5-7.5 mg/ml or about 5 mg/mL;
(ii) D-lactic acid or a pharmaceutically acceptable salt thereof, at a concentration of 100-500 mM, 100-300 mM, 100-200 mM, 100-150 mM, about 100 mM or about 200 mM;
(iii) sucrose at a concentration of 200-1200 mM, such as 300-800 mM;
(iv) water, e.g. water for injection;
(v) optionally an inorganic salt containing a metal cation;
(vi) optionally an antioxidant;
(vii) optionally a non-ionic surfactant; and
(viii) optionally a preservative;
   wherein the osmolarity of the composition is 400-1250 mOsm/L, e.g. 450-1250 mOsm/L, or 550-1250 mOsm/L; and
   wherein the pH of the composition is in the range 4.0 to 6.0, such as about 5.0.

In one embodiment, there is provided a storage stable aqueous solution composition consisting of, or consisting essentially of:
(i) vancomycin or a pharmaceutically acceptable salt thereof, at a concentration of 1-10 mg/mL, e.g. 2.5-7.5 mg/ml or about 5 mg/mL;
(ii) D-lactic acid or a pharmaceutically acceptable salt thereof, at a concentration of 100-500 mM, 100-300 mM, 100-200 mM, 100-150 mM, about 100 mM or about 200 mM;
(iii) a mixture of two polyols selected from the group consisting of propylene glycol, sucrose, glycerol, trehalose, lactose, glucose, sorbitol and mannitol, at a combined total concentration of 200-1200 mM, such as 300-800 mM;
(iv) water, e.g. water for injection;
(v) optionally an inorganic salt containing a metal cation;
(vi) optionally an antioxidant;
(vii) optionally a non-ionic surfactant; and
(viii) optionally a preservative;
   wherein the osmolarity of the composition is 400-1250 mOsm/L, e.g. 450-1250 mOsm/L, or 550-1250 mOsm/L; and
   wherein the pH of the composition is in the range 4.0 to 6.0, such as about 5.0.

In one embodiment, there is provided a storage stable aqueous solution composition consisting of, or consisting essentially of:
(i) vancomycin or a pharmaceutically acceptable salt thereof, at a concentration of 1-10 mg/mL, e.g. 2.5-7.5 mg/mL or about 5 mg/ml;
(ii) D-lactic acid or a pharmaceutically acceptable salt thereof, at a concentration of 100-500 mM, 100-300 mM, 100-200 mM, 100-150 mM, about 100 mM or about 200 mM;
(iii) a mixture of propylene glycol at a concentration of 10-160 mM, e.g. 10-150 mM or 10-125 mM, and a polyol selected from the group consisting of sucrose, glycerol, trehalose, lactose, glucose, sorbitol and mannitol, at a concentration of 40-1190 mM, e.g. 50-1190 mM or 75-1190 mM; wherein the combined total concentration of propylene glycol and polyol selected from the group consisting of sucrose, glycerol, trehalose, lactose, glucose, sorbitol and mannitol is 200-1200 mM, such as 300-800 mM;
(iv) water, e.g. water for injection;
(v) optionally an inorganic salt containing a metal cation;
(vi) optionally an antioxidant;
(vii) optionally a non-ionic surfactant; and
(viii) optionally a preservative;
   wherein the osmolarity of the composition is 400-1250 mOsm/L, e.g. 450-1250 mOsm/L, or 550-1250 mOsm/L; and
   wherein the pH of the composition is in the range 4.0 to 6.0, such as about 5.0.

In one embodiment, there is provided a storage stable aqueous solution composition consisting of, or consisting essentially of:
(i) vancomycin or a pharmaceutically acceptable salt thereof, at a concentration of 1-10 mg/mL, e.g. 2.5-7.5 mg/mL or about 5 mg/mL;
(ii) DL-lactic acid or a pharmaceutically acceptable salt thereof, at a concentration of 200-1000 mM, 200-600 mM, 200-400 mM, 200-300 mM, about 200 mM or about 400 mM;
(iii) a polyol selected from the group consisting of propylene glycol, sucrose, glycerol, trehalose, lactose, glucose, sorbitol and mannitol, or a mixture thereof, at a total concentration of 200-1200 mM, such as 300-800 mM;
(iv) water, e.g. water for injection;
(v) optionally an inorganic salt containing a metal cation;
(vi) optionally an antioxidant;
(vii) optionally a non-ionic surfactant; and
(viii) optionally a preservative;
   wherein the osmolarity of the composition is 400-1250 mOsm/L, e.g. 450-1250 mOsm/L, or 550-1250 mOsm/L; and
   wherein the pH of the composition is in the range 4.0 to 6.0, such as about 5.0.

In one embodiment, there is provided a storage stable aqueous solution composition consisting of, or consisting essentially of:
(i) vancomycin or a pharmaceutically acceptable salt thereof, at a concentration of 1-10 mg/mL, e.g. 2.5-7.5 mg/mL or about 5 mg/mL;
(ii) DL-lactic acid or a pharmaceutically acceptable salt thereof, at a concentration of 200-1000 mM, 200-600 mM, 200-400 mM, 200-300 mM, about 200 mM or about 400 mM;
(iii) propylene glycol at a concentration of 660-1200 mM, e.g. 700-1200 mM, 750-1200 mM, 800-1200 mM or 850-1200 mM;
(iv) water, e.g. water for injection;
(v) optionally an inorganic salt containing a metal cation;
(vi) optionally an antioxidant;
(vii) optionally a non-ionic surfactant; and
(viii) optionally a preservative;
   wherein the osmolarity of the composition is 400-1250 mOsm/L, e.g. 450-1250 mOsm/L, or 550-1250 mOsm/L; and
   wherein the pH of the composition is in the range 4.0 to 6.0, such as about 5.0.

In one embodiment, there is provided a storage stable aqueous solution composition consisting of, or consisting essentially of:
(i) vancomycin or a pharmaceutically acceptable salt thereof, at a concentration of 1-10 mg/mL, e.g. 2.5-7.5 mg/mL or about 5 mg/mL;
(ii) DL-lactic acid or a pharmaceutically acceptable salt thereof, at a concentration of 200-1000 mM, 200-600 mM, 200-400 mM, 200-300 mM, about 200 mM or about 400 mM;
(iii) sucrose at a concentration of 200-1200 mM, such as 300-800 mM;
(iv) water, e.g. water for injection;
(v) optionally an inorganic salt containing a metal cation;
(vi) optionally an antioxidant;
(vii) optionally a non-ionic surfactant; and
(viii) optionally a preservative;
   wherein the osmolarity of the composition is 400-1250 mOsm/L, e.g. 450-1250 mOsm/L, or 550-1250 mOsm/L; and
   wherein the pH of the composition is in the range 4.0 to 6.0, such as about 5.0.

In one embodiment, there is provided a storage stable aqueous solution composition consisting of, or consisting essentially of:
(i) vancomycin or a pharmaceutically acceptable salt thereof, at a concentration of 1-10 mg/mL, e.g. 2.5-7.5 mg/mL or about 5 mg/mL;
(ii) DL-lactic acid or a pharmaceutically acceptable salt thereof, at a concentration of 200-1000 mM, 200-600 mM, 200-400 mM, 200-300 mM, about 200 mM or about 400 mM;
(iii) a mixture of two polyols selected from the group consisting of propylene glycol, sucrose, glycerol, trehalose, lactose, glucose, sorbitol and mannitol, at a combined total concentration of 200-1200 mM, such as 300-800 mM;
(iv) water, e.g. water for injection;
(v) optionally an inorganic salt containing a metal cation;
(vi) optionally an antioxidant;
(vii) optionally a non-ionic surfactant; and
(viii) optionally a preservative;
   wherein the osmolarity of the composition is 400-1250 mOsm/L, e.g. 450-1250 mOsm/L, or 550-1250 mOsm/L; and
   wherein the pH of the composition is in the range 4.0 to 6.0, such as about 5.0.

In one embodiment, there is provided a storage stable aqueous solution composition consisting of, or consisting essentially of:
(i) vancomycin or a pharmaceutically acceptable salt thereof, at a concentration of 1-10 mg/mL, e.g. 2.5-7.5 mg/ml or about 5 mg/mL;
(ii) DL-lactic acid or a pharmaceutically acceptable salt thereof, at a concentration of 200-1000 mM, 200-600 mM, 200-400 mM, 200-300 mM, about 200 mM or about 400 mM;
(iii) a mixture of propylene glycol at a concentration of 10-160 mM, e.g. 10-150 mM or 10-125 mM, and a polyol selected from the group consisting of sucrose, glycerol, trehalose, lactose, glucose, sorbitol and mannitol, at a concentration of 40-1190 mM, e.g. 50-1190 mM or 75-1190 mM; wherein the combined total concentration of propylene glycol and polyol selected from the group consisting of sucrose, glycerol, trehalose, lactose, glucose, sorbitol and mannitol is 200-1200 mM, such as 300-800 mM;
(iv) water, e.g. water for injection;
(v) optionally an inorganic salt containing a metal cation;
(vi) optionally an antioxidant;
(vii) optionally a non-ionic surfactant; and
(viii) optionally a preservative;
   wherein the osmolarity of the composition is 400-1250 mOsm/L, e.g. 450-1250 mOsm/L, or 550-1250 mOsm/L; and
   wherein the pH of the composition is in the range 4.0 to 6.0, such as about 5.0.

As used herein when referring to the composition of the invention, the term “consists of” means that no further components are included in the composition other than those listed. As used herein when referring to the composition of the invention, the term “consists essentially of” means that specific further components can be present, but said components do not materially affect the essential characteristics of the composition, and are typically present in a de minimis amount.

Suitably the composition of the invention remains as a clear solution (e.g. as measured according to the Visual Assessment Method in the General Methods) following storage at 2-8° C. for an extended period of time, such as at least 6 months, at least 12 months, at least 18 months or at least 24 months.

Suitably the composition of the invention remains as a clear solution (e.g. as measured according to the Visual Assessment Method in the General Methods) following storage at 25° C. for at least 1 week, such as at least 2 weeks, at least 4 weeks or at least 8 weeks.

Suitably the composition of the invention comprises no more than 12% total impurities with no individual impurity being >4% (by total weight of vancomycin or pharmaceutically acceptable salt thereof in the composition) as set out in the US Pharmacopeial Monograph (Vancomycin hydrochloride for Injection, USP29), following storage at 2-8° C. for an extended period of time, such as at least 6 months, at least 12 months, at least 18 months or at least 24 months.

Suitably the composition of the invention comprises no more than 7% total impurities with no individual impurity being >4% (by total weight of vancomycin or pharmaceutically acceptable salt thereof in the composition) as set out in the European Pharmacopeial Monograph (Vancomycin hydrochloride, EP 6.0), following storage at 2-8° C. for an extended period of time, such as at least 6 months, at least 12 months, at least 18 months or at least 24 months.

Suitably the composition of the invention comprises no more than 12% total impurities with no individual impurity being >4% (by total weight of vancomycin or pharmaceutically acceptable salt thereof in the composition) as set out in the US Pharmacopeial Monograph (Vancomycin hydrochloride for Injection, USP29), following storage at 25° C. for at least 1 week, such as at least 2 weeks, at least 4 weeks or at least 8 weeks.

Suitably the composition of the invention comprises no more than 7% total impurities with no individual impurity being >4% (by total weight of vancomycin or pharmaceutically acceptable salt thereof in the composition) as set out in the European Pharmacopeial Monograph (Vancomycin hydrochloride, EP 6.0), following storage at 25° C. for at least 1 week, such as at least 2 weeks, at least 4 weeks or at least 8 weeks.

In one embodiment, the composition of the invention is a composition for use in therapy. In one embodiment, the composition of the invention is a pharmaceutical composition.

Vancomycin (in particular vancomycin hydrochloride) is indicated inter alia for the treatment of bacterial infection, in particular for the treatment of serious or severe bacterial infections caused by susceptible strains of methicillin-resistant (beta-lactam-resistant) staphylococci. It is indicated for penicillin-allergic patients, for patients who cannot receive or who have failed to respond to other drugs, including the penicillins or cephalosporins, and for bacterial infections caused by vancomycin-susceptible organisms that are resistant to other antimicrobial drugs. Vancomycin (in particular vancomycin hydrochloride) is indicated for initial therapy when methicillin-resistant staphylococci are suspected, but after susceptibility data are available, therapy should be adjusted accordingly.

Vancomycin (in particular vancomycin hydrochloride) is also effective in the treatment of staphylococcal endocarditis. Its effectiveness has been documented in other bacterial infections due to staphylococci, including septicaemia, bone infections, lower respiratory tract infections, skin and skin structure infections. When staphylococcal infections are localized and purulent, antibiotics are used as adjuncts to appropriate surgical measures. Vancomycin (in particular vancomycin hydrochloride) has been reported to be effective alone or in combination with an aminoglycoside for endocarditis caused by Streptococcus viridans or Streptococcus bovis. For endocarditis caused by enterococci (e.g., Enterococcus faecalis), vancomycin (in particular vancomycin hydrochloride) has been reported to be effective only in combination with an aminoglycoside. Vancomycin has been reported to be effective for the treatment of diphtheroid endocarditis. Vancomycin has been used successfully in combination with either rifampin, an aminoglycoside, or both in early-onset prosthetic valve endocarditis caused by Staphylococcus epidermidis or diphtheroids.

Accordingly, in one embodiment is provided a composition as described herein above for use in treating bacterial infection. In one embodiment is provided a composition as described hereinabove for use in the manufacture of a medicament for treating bacterial infection. In one embodiment is provided a method of treating bacterial infection, which comprises administering to a patient, particularly a human patient, in need thereof a therapeutically effective amount of a composition as described hereinabove.

In one embodiment, the bacterial infection is caused by gram positive bacteria, and is suitably selected from the group consisting of Clostridium difficile, Listeria monocytogenes, Streptococcus pyogenes, pneumoniae, Streptococcus Streptococcus agalactiae, Streptococcus viridans, Streptococcus bovis, Staphylococcus epidermidis, multidrug resistant Staphylococcus epidermidis, Staphylococcus aureus, methicillin-resistant Staphylococcos aureus, Actinomyces species, Lactobacillus species, Enterococcus faecalis, and diphtheroids.

The vancomycin compositions of the present invention can be used as a sole therapy, or in combination with one or more other therapeutic agents. In one embodiment, the further therapeutic agent(s) is/are antibiotics, e.g. selected from the group consisting of rifampicin, an aminoglycoside (such as gentamicin), and a beta-lactam antibiotic (such as amoxicillin).

In one embodiment is provided a composition as described hereinabove for use in treating bacterial infection, with an antibiotic, e.g. selected from the group consisting of rifampicin, an aminoglycoside (such as gentamicin), and a beta-lactam antibiotic (such as amoxicillin). In one embodiment is provided an antibiotic, e.g. selected from the group consisting of rifampicin, an aminoglycoside (such as gentamicin), and a beta-lactam antibiotic (such as amoxicillin) for use in treating bacterial infection, with a composition as described hereinabove.

All embodiments described above with respect to the aqueous solution composition apply equally to methods and uses of the invention.

There is also provided a container, containing a dose or a plurality of doses of the composition as described hereinabove. The container is suitably an intravenous (IV) solution bag or bottle. When the container is an IV solution bag, it is suitably constructed from plastic such as PVC, polyolefin, polypropylene, a blend of PO and PP (polyolefin blend), polyurethane, or a material that is substantially free of PVC, plasticizers, adhesives and latex, such as DEHP (di(2-ethylhexyl) phthalate). When the container is an IV solution bottle, it can be made from plastic (as described above for the IV solution bag) or can be made from glass, e.g. it is a type 1 glass bottle. In one embodiment, the volume of the IV solution bag or bottle is 50 ml to 1 L, e.g. 50 mL, 100 mL, 150 mL, 200 mL, 250 ml, 300 mL, 400 mL, 500 mL, 750 mL or 1 L.

Suitably the composition is free of visible particles and is a colourless to slightly yellow solution. Suitably, once opened, the composition is stable for at least 24 hours at 2-8° C. and at least 3 hours at 25° C. and room light.

The invention is further defined by the following clauses:

Clause 1. A storage stable aqueous solution composition comprising:
vancomycin or a pharmaceutically acceptable salt thereof, at a concentration of 1-10 mg/mL;
D-lactic acid or a pharmaceutically acceptable salt thereof; and
a polyol selected from the group consisting of propylene glycol, sucrose, glycerol, trehalose, lactose, glucose, sorbitol and mannitol, or a mixture thereof;
wherein the osmolarity of the composition is 400-1250 mOsm/L.

Clause 2. The storage stable aqueous solution composition according to clause 1, wherein the vancomycin form is vancomycin hydrochloride.

Clause 3. The storage stable aqueous solution composition according to clause 1 or clause 2, wherein the concentration of vancomycin or a pharmaceutically acceptable salt thereof is 2.5-7.5 mg/ml or about 5 mg/mL.

Clause 4. The storage stable aqueous solution composition according to any one of clauses 1 to 3, wherein the composition is substantially free of, or free of L-lactic acid and salts thereof.

Clause 5. The storage stable aqueous solution composition according to any one of clauses 1 to 4, comprising sodium D-lactate, calcium D-lactate or potassium D-lactate, in particular sodium D-lactate.

Clause 6. The storage stable aqueous solution composition according to any one of clauses 1 to 3, or clause 5, wherein the source of the D-lactic acid or a pharmaceutically acceptable salt thereof is DL-lactic acid or a pharmaceutically acceptable salt thereof.

Clause 7. The storage stable aqueous solution composition according to clause 6, comprising sodium DL-lactate, calcium DL-lactate or potassium DL-lactate, in particular sodium DL-lactate.

Clause 8. The storage stable aqueous solution composition according to any one of clauses 1 to 7, wherein the concentration of D-lactic acid or a pharmaceutically acceptable salt thereof is 100-500 mM, e.g. 100-300 mM and in particular is 100-200 mM e.g. about 100 mM or about 200 mM.

Clause 9. The storage stable aqueous solution composition according to clause 6 or clause 7, wherein the concentration of DL-lactic acid or a pharmaceutically acceptable salt thereof is 200-1000 mM, e.g. 200-600 mM and in particular is 200-400 mM e.g. about 200 mM or about 400 mM.

Clause 10. The storage stable aqueous solution composition according to any one of clauses 1 to 9, wherein the total concentration of polyol in the composition is 200-1200 mM, such as 300-800 mM.

Clause 11. The storage stable aqueous solution composition according to any one of clauses 1 to 10, wherein the composition comprises a single polyol selected from the group consisting of propylene glycol, sucrose, glycerol, trehalose, lactose, glucose, sorbitol and mannitol.

Clause 12. The storage stable aqueous solution composition according to clause 11, wherein the single polyol is propylene glycol, which is suitably present in the composition at a concentration of 660-1200 mM, e.g. 700-1200 mM, 750-1200 mM, 800-1200 mM or 850-1200 mM.

Clause 13. The storage stable aqueous solution composition according to clause 11, wherein the single polyol is glycerol, which is suitably present in the composition at a concentration of 200-1200 mM, such as 300-800 mM.

Clause 14. The storage stable aqueous solution composition according to clause 11, wherein the single polyol is sucrose, which is suitably present in the composition at a concentration of 200-1200 mM, such as 300-800 mM.

Clause 15. The storage stable aqueous solution composition according to clause 11, wherein the single polyol is mannitol, which is suitably present in the composition at a concentration of 200-1200 mM, such as 300-800 mM.

Clause 16. The storage stable aqueous solution composition according to any one of clauses 1 to 10, wherein the composition comprises a mixture of two polyols selected from the group consisting of propylene glycol, sucrose, glycerol, trehalose, lactose, glucose, sorbitol and mannitol.

Clause 17. The storage stable aqueous solution composition according to any one of clauses 1 to 11 or clause 16, wherein the or each polyol is selected from the group consisting of sucrose, glycerol, trehalose, lactose, glucose, sorbitol and mannitol.

Clause 18. The storage stable aqueous solution composition according to any one of clauses 1 to 11 or clause 16, wherein the or each polyol is selected from the group consisting of propylene glycol, sucrose, trehalose, lactose, glucose, sorbitol and mannitol.

Clause 19. The storage stable aqueous solution composition according to any one of clauses 1 to 11 or clause 16, wherein the or each polyol is selected from the group consisting of sucrose, trehalose, lactose, glucose, sorbitol and mannitol.

Clause 20. The storage stable aqueous solution composition according to clause 16, wherein the composition comprises a mixture of propylene glycol and a polyol selected from the group consisting of sucrose, glycerol, trehalose, lactose, glucose, sorbitol and mannitol; wherein the propylene glycol is suitably present at a concentration of 10-160 mM, e.g. 10-150 mM or 10-125 mM; and the polyol selected from the group consisting of sucrose, glycerol, trehalose, lactose, glucose, sorbitol and mannitol is suitably present at a concentration of 40-1190 mM, e.g. 50-1190 mM or 75-1190 mM.

Clause 21. The storage stable aqueous solution composition according to clause 20, wherein the combined total concentration of propylene glycol and polyol selected from the group consisting of sucrose, glycerol, trehalose, lactose, glucose, sorbitol and mannitol is 200-1200 mM, such as 300-800 mM.

Clause 22. The storage stable aqueous solution composition according to clause 20, wherein the composition comprises a mixture of propylene glycol and glycerol, wherein the propylene glycol is suitably present in the composition at a concentration of 10-160 mM, e.g. 10-150 mM or 10-125 mM, and the glycerol is suitably present in the composition at a concentration of 40-1190 mM, e.g. 50-1190 mM or 75-1190 mM.

Clause 23. The storage stable aqueous solution composition according to clause 22, wherein the combined total concentration of propylene glycol and glycerol is 200-1200 mM, such as 300-800 mM.

Clause 24. The storage stable aqueous solution composition according to clause 16, wherein the composition comprises a mixture of propylene glycol and sucrose, wherein the propylene glycol is suitably present in the composition at a concentration of 10-160 mM, e.g. 10-150 mM or 10-125 mM, and the sucrose is suitably present in the composition at a concentration of 40-1190 mM, e.g. 50-1190 mM or 75-1190 mM.

Clause 25. The storage stable aqueous solution composition according to clause 20, wherein the combined total concentration of propylene glycol and sucrose is 200-1200 mM, such as 300-800 mM.

Clause 26. The storage stable aqueous solution composition according to clause 16, wherein the composition comprises a mixture of propylene glycol and mannitol, wherein the propylene glycol is suitably present in the composition at a concentration of 10-160 mM, e.g. 10-150 mM or 10-125 mM, and the mannitol is suitably present in the composition at a concentration of 40-1190 mM, e.g. 50-1190 mM or 75-1190 mM.

Clause 27. The storage stable aqueous solution composition according to clause 26, wherein the combined total concentration of propylene glycol and mannitol is 200-1200 mM, such as 300-800 mM.

Clause 28. The storage stable aqueous solution composition according to any one of clauses 1 to 27, wherein the molar ratio of vancomycin or a pharmaceutically acceptable salt thereof, to polyol is between 1:100 and 1:1000, e.g. between 1:100 and 1:500, in particular between 1:100 and 1:250.

Clause 29. The storage stable aqueous solution composition according to any one of clauses 1 to 28, wherein the osmolarity of the composition is 410-1250 mOsm/L, 425-1250 mOsm/L, 450-1250 mOsm/L, 500-1250 mOsm/L, 550-1250 mOsm/L, 600-1250 mOsm/L, 700-1250 mOsm/L, 400-1100 mOsm/L, 410-1100 mOsm/L, 425-1100 mOsm/L, 450-1100 mOsm/L, 500-1100 mOsm/L, 550-1100 mOsm/L, 600-1100 mOsm/L, 700-1100 mOsm/L, 400-1000 mOsm/L, 410-1000 mOsm/L, 425-1000 mOsm/L, 450-1000 mOsm/L, 500-1000 mOsm/L, 550-1000 mOsm/L, 600-1000 mOsm/L, 700-1000 mOsm/L, 400-800 mOsm/L, 410-800 mOsm/L, 425-800 mOsm/L, 450-800 mOsm/L, 500-800 mOsm/L, 550-800 mOsm/L, 600-800 mOsm/L, 700-800 mOsm/L, 400-600 mOsm/L, 410-600 mOsm/L, 425-600 mOsm/L, 450-600 mOsm/L, 500-600 mOsm/L or 500-600 mOsm/L.

Clause 30. The storage stable aqueous solution composition according to any one of clauses 1 to 29, comprising an inorganic salt containing a metal cation, especially a Group 1 or Group 2 metal cation.

Clause 31. The storage stable aqueous solution composition according to clause 30, wherein the inorganic salt containing a metal cation is selected from the group consisting of sodium chloride, calcium chloride, sodium oxamate and sodium sulphate.

Clause 32. The storage stable aqueous solution composition according to clause 30 or clause 31, wherein the concentration of inorganic salt containing a metal cation is 1-500 mM, e.g. 50-250 mM or 75-150 mM.

Clause 33. The storage stable aqueous solution composition according to any one of clauses 1 to 32, wherein the pH of the composition is in the range 4.0 to 6.0, such as about 5.0.

Clause 34. The storage stable aqueous solution composition according to any one of clauses 1 to 33, further comprising an antioxidant.

Clause 35. The storage stable aqueous solution composition according to clause 34, wherein the antioxidant is selected from the group consisting of monothioglycerol, butylated hydroxyanisole, glutathione (reduced), ascorbate, cysteine and methionine, and is suitably methionine.

Clause 36. The storage stable aqueous solution composition according to clause 34 or clause 35, wherein the antioxidant is present at a concentration of 1-100 mM, e.g. 10-50 mM or 25-50 mM.

Clause 37. The storage stable aqueous solution composition according to any one of clauses 1 to 36, wherein the composition is substantially free of, or free of amino acids.

Clause 38. The storage stable aqueous solution composition according to any one of clauses 1 to 37, further comprising a non-ionic surfactant.

Clause 39. The storage stable aqueous solution composition according to clause 38, wherein the non-ionic surfactant is selected from the group consisting of an alkyl glycoside, a polysorbate, an alkyl ether of polyethylene glycol, a block copolymer of polyethylene glycol and polypropylene glycol, and an alkylphenyl ether of polyethylene glycol.

Clause 40. The storage stable aqueous solution composition according to clause 39, wherein the non-ionic surfactant is an alkyl glycoside which is suitably selected from the group consisting of dodecyl maltoside, dodecyl glucoside, octyl glucoside, octyl maltoside, decyl glucoside, decyl maltoside, decyl glucopyranoside, tridecyl glucoside, tridecyl maltoside, tetradecyl glucoside, tetradecyl maltoside, hexadecyl glucoside, hexadecyl maltoside, sucrose monooctanoate, sucrose monodecanoate, sucrose monododecanoate, sucrose monotridecanoate, sucrose monotetradecanoate and sucrose monohexadecanoate; and in particular is dodecyl maltoside or decyl glucopyranoside, especially dodecyl maltoside.

Clause 41. The storage stable aqueous solution composition according to clause 39, wherein the non-ionic surfactant is a polysorbate which is suitably polysorbate 20 or polysorbate 80.

Clause 42. The storage stable aqueous solution composition according to clause 39, wherein the non-ionic surfactant is an alkyl ether of polyethylene glycol which is suitably selected from the group consisting of polyethylene glycol (2) hexadecyl ether (Brij 52), polyethylene glycol (2) oleyl ether (Brij 93), polyethylene glycol (2) dodecyl ether (Brij L4), polyethylene glycol (4) lauryl ether (Brij 30), polyethylene glycol (10) lauryl ether (Brij 35), polyethylene glycol (20) hexadecyl ether (Brij 58) and polyethylene glycol (10) stearyl ether (Brij 78).

Clause 43. The storage stable aqueous solution composition according to clause 39, wherein the non-ionic surfactant is a block copolymer of polyethylene glycol and polypropylene glycol, which is suitably selected from the group consisting of poloxamer 188, poloxamer 407, poloxamer 171 or poloxamer 185.

Clause 44. The storage stable aqueous solution composition according to clause 39, wherein the non-ionic surfactant is alkylphenyl ether of polyethylene glycol, which is suitably 4-(1,1,3,3-tetramethylbutyl)phenyl-polyethylene glycol.

Clause 45. The storage stable aqueous solution composition according to any one of clauses 38 to 44, wherein the non-ionic surfactant is present at a concentration of 1-5000 μg/mL, 1-1000 μg/mL, such as 5-500 μg/mL, 10-400 μg/mL, 20-400 μg/mL, 50-400 μg/mL, 10-300 μg/mL, 20-300 μg/mL, 50-300 μg/mL, 10-200 μg/mL, 20-200 μg/mL, 50-200 μg/mL, 10-100 μg/mL, 20-100 μg/mL, 50-100 μg/mL or around 50 μg/mL.

Clause 46. The storage stable aqueous solution composition according to any one of clauses 1 to 45, further comprising a preservative such as a phenolic or benzylic preservative.

Clause 47. The storage stable aqueous solution composition according to clause 46, wherein the phenolic or benzylic preservative is selected from the group consisting of phenol, m-cresol, chlorocresol, benzyl alcohol, propyl paraben and methyl paraben.

Clause 48. The storage stable aqueous solution composition according to any one of clauses 1 to 47, comprising at least 80% (v/v) water, such as at least 70% (v/v), at least 60% (v/v), at least 55% (v/v), at least 50% (v/v), at least 45% (v/v), at least 40% (v/v), at least 35% (v/v), at least 30% (v/v), at least 25% (v/v), at least 20% (v/v), at least 15% (v/v), at least 10% (v/v), at least 5% (v/v) or at least 2.5% (v/v) of water, e.g. sterile water for injection or bacteriostatic water for injection.

Clause 49. The storage stable aqueous solution composition according to clause 1, comprising, consisting essentially of, or consisting of:
vancomycin or a pharmaceutically acceptable salt thereof, at a concentration of 1-10 mg/mL, e.g. 2.5-7.5 mg/mL or about 5 mg/mL;
D-lactic acid or a pharmaceutically acceptable salt thereof, at a concentration of 100-500 mM, 100-300 mM, 100-200 mM, 100-150 mM, about 100 mM or about 200 mM; or DL-lactic acid or a pharmaceutically acceptable salt thereof, at a concentration of 200-1000 mM, 200-600 mM, 200-400 mM, 200-300 mM, about 200 mM or about 400 mM;
a polyol selected from the group consisting of propylene glycol, sucrose, glycerol, trehalose, lactose, glucose, sorbitol and mannitol, or a mixture thereof, at a total concentration of 200-1200 mM, such as 300-800 mM; and
optionally an inorganic salt containing a metal cation;
   wherein the osmolarity of the composition is 400-1250 mOsm/L, e.g. 450-1250 mOsm/L, or 550-1250 mOsm/L; and
   wherein the pH of the composition is in the range 4.0 to 6.0, such as about 5.0.

Clause 50. The storage stable aqueous solution composition according to clause 1, comprising, consisting essentially of, or consisting of:
vancomycin or a pharmaceutically acceptable salt thereof, at a concentration of 1-10 mg/mL, e.g. 2.5-7.5 mg/mL or about 5 mg/mL;
D-lactic acid or a pharmaceutically acceptable salt thereof, at a concentration of 100-500 mM, 100-300 mM, 100-200 mM, 100-150 mM, about 100 mM or about 200 mM; or DL-lactic acid or a pharmaceutically acceptable salt thereof, at a concentration of 200-1000 mM, 200-600 mM, 200-400 mM, 200-300 mM, about 200 mM or about 400 mM;
a mixture of two polyols selected from the group consisting of propylene glycol, sucrose, glycerol, trehalose, lactose, glucose, sorbitol and mannitol, at a combined total concentration of 200-1200 mM, such as 300-800 mM; and
optionally an inorganic salt containing a metal cation;
   wherein the osmolarity of the composition is 400-1250 mOsm/L, e.g. 450-1250 mOsm/L, or 550-1250 mOsm/L; and
   wherein the pH of the composition is in the range 4.0 to 6.0, such as about 5.0.

Clause 51. The storage stable aqueous solution composition according to clause 1, comprising, consisting essentially of, or consisting of:
vancomycin or a pharmaceutically acceptable salt thereof, at a concentration of 1-10 mg/mL, e.g. 2.5-7.5 mg/mL or about 5 mg/mL;
D-lactic acid or a pharmaceutically acceptable salt thereof, at a concentration of 100-500 mM, 100-300 mM, 100-200 mM, 100-150 mM, about 100 mM or about 200 mM; or DL-lactic acid or a pharmaceutically acceptable salt thereof, at a concentration of 200-1000 mM, 200-600 mM, 200-400 mM, 200-300 mM, about 200 mM or about 400 mM;
a mixture of propylene glycol at a concentration of 10-160 mM, e.g. 10-150 mM or 10-125 mM, and a polyol selected from the group consisting of sucrose, glycerol, trehalose, lactose, glucose, sorbitol and mannitol, at a concentration of 40-1190 mM, e.g. 50-1190 mM or 75-1190 mM; wherein the combined total concentration of propylene glycol and polyol selected from the group consisting of sucrose, glycerol, trehalose, lactose, glucose, sorbitol and mannitol is 200-1200 mM, such as 300-800 mM; and
optionally an inorganic salt containing a metal cation;
   wherein the osmolarity of the composition is 400-1250 mOsm/L, e.g. 450-1250 mOsm/L, or 550-1250 mOsm/L; and
   wherein the pH of the composition is in the range 4.0 to 6.0, such as about 5.0.

Clause 52. The storage stable aqueous solution composition according to any one of clauses 1 to 51, which is a pharmaceutical composition.

Clause 53. The storage stable aqueous solution composition according to any one of clauses 1 to 51, or the pharmaceutical composition according to clause 52, for use in therapy.

Clause 54. The storage stable aqueous solution composition or the pharmaceutical composition according to clause 53, for use in treating bacterial infection.

Clause 55. A method of treating bacterial infection which comprises administering to a patient in need thereof a therapeutically effective amount of a storage stable aqueous solution composition according to any one of clauses 1 to 51, or a pharmaceutical composition according to clause 52.

Clause 56. The storage stable aqueous solution composition for use according to clause 54, or the method according to clause 55, wherein the bacterial infection is caused by gram positive bacteria, and is suitably selected from the group consisting of Clostridium difficile, Listeria monocytogenes, Streptococcus pyogenes, Streptococcus pneumoniae, Streptococcus agalactiae, Streptococcus viridans, Streptococcus bovis, Staphylococcus epidermidis, multidrug resistant Staphylococcus epidermidis, Staphylococcus aureus, methicillin-resistant Staphylococcos aureus, Actinomyces species, Lactobacillus species, Enterococcus faecalis, and diphtheroids.

Clause 57. The storage stable aqueous solution composition for use or the method according to any one of clauses 54 to 56, for use in combination with one or more further therapeutic agent(s).

Clause 58. The storage stable aqueous solution composition for use or the method according to clause 57, wherein the one or more further therapeutic agent(s) is/are selected from the group consisting of rifampicin, an aminoglycoside (such as gentamicin), and a beta-lactam antibiotic (such as amoxicillin).

Clause 59. A ready-to-administer intravenous (IV) solution container such as a bag or bottle containing the storage stable aqueous solution composition according to any one of clauses 1 to 52.

### EXAMPLES

### General Methods

### Reversed-Phase Chromatography Method (RP-HPLC)

High performance reverse phase chromatography is performed using the Thermo-Scientific Ultimate 3000 HPLC with Diode Array Detector (DAD3000) with a Zorbax Stable Bond C18 250 mm by 4.6 mm, 5 μm particle size and 80 Å pore size. Mobile Phase A is a mixture of triethylamine buffer (mix 4 mL of triethylamine and 2000 ml of water, and adjust with phosphoric acid to a pH of 3.2), acetonitrile and tetrahydrofuran in volume per volume ratio of 92:7:1. Mobile Phase B is a mixture of triethylamine buffer, acetonitrile, and tetrahydrofuran in volume per volume ratio of 70:29:1. The sample comprising vancomycin (or pharmaceutically acceptable salt thereof) is bound in Mobile Phase A and eluted using a gradient of Mobile Phase A and Mobile Phase B. The sample volume is 20 microlitres, the flow rate is 1.0 mL/min, with 284 nm UV detection. All analyses are performed at 25° C.

### Visual Assessment Method

Visible particles are suitably detected using the 2.9.20. European Pharmacopoeia Monograph (Particulate Contamination: Visible Particles). The apparatus required consists of a viewing station comprising:
a matt black panel of appropriate size held in a vertical position.
a non-glare white panel of appropriate size held in a vertical position next to the black panel
an adjustable lampholder fitted with a suitable, shaded, white-light source and with a suitable light diffuser (a viewing illuminator containing two 13 W fluorescent tubes, each 525 mm in length, is suitable). The intensity of illumination at the viewing point is maintained between 2000 lux and 3750 lux.

Any adherent labels are removed from the container and the outside washed and dried. The container is gently swirled or inverted, ensuring that air bubbles are not introduced, and observed for about 5 s in front of the white panel. The procedure is repeated in front of the black panel. The presence of any particles is recorded. The visual scores are ranked as follows:
Visual score 1: Clear solution, virtually free of particles
Visual score 2: ˜5 very small particles
Visual score 3: ˜10-20 very small particles
Visual score 4:20-50 particles, including larger particles
Visual score 5: >50 particles, including larger particles

Whilst the particles in samples with visual scores 4 and 5 are clearly detectable on casual visual assessment under normal light, samples with visual score 1-3 generally appear as clear solutions on the same assessment. Samples with visual scores 1-3 are considered to be “Pass”; samples with visual score 4-5 are considered to be “Fail”.

### Osmolarity of Compositions

A number of the vancomycin compositions prepared in Examples 2-8 have osmolarity in excess of 1250 mOsm/L. These compositions illustrate aspects of the invention, particularly stability trends associated with use of certain composition components, even though they are not embodiments of the invention as such due to their high osmolarity.

### Example 1: Composition Examples

The following example aqueous compositions may be prepared.

### Example A

| Vancomycin hydrochloride | 5 | mg/mL |
|---|---|---|
| D-lactic acid | 100 | mM |
| Propylene glycol | 1000 | mM |
| pH | 5.0 | |
| Osmolarity | 1190 | mOsm/L |

### Example B

| Vancomycin hydrochloride | 5 | mg/mL |
|---|---|---|
| Sodium D-lactate | 200 | mM |
| Propylene glycol | 800 | mM |
| pH | 5.0 | |
| Osmolarity | 1200 | mOsm/L |

### Example C

| Vancomycin hydrochloride | 5 | mg/mL |
|---|---|---|
| D-lactic acid | 200 | mM |
| Sucrose | 300 | mM |
| pH | 5.0 | |
| Osmolarity | 680 | mOsm/L |

### Example D

| Vancomycin hydrochloride | 5 | mg/mL |
|---|---|---|
| Sodium D-Lactate | 100 | mM |
| Sucrose | 400 | mM |
| pH | 5.0 | |
| Osmolarity | 600 | mOsm/L |

### Example E

| Vancomycin hydrochloride | 5 | mg/mL |
|---|---|---|
| DL-lactic acid | 200 | mM |
| Propylene glycol | 800 | mM |
| pH | 5.0 | |
| Osmolarity | 1180 | mOsm/L |

### Example F

| Vancomycin hydrochloride | 5 | mg/mL |
|---|---|---|
| Sodium DL-lactate | 400 | mM |
| Sucrose | 300 | mM |
| pH | 5.0 | |
| Osmolarity | 1100 | mOsm/L |

### Example G

| Vancomycin hydrochloride | 5 | mg/mL |
|---|---|---|
| Sodium DL-lactate | 200 | mM |
| Sucrose | 350 | mM |
| Propylene glycol | 150 | mM |
| pH | 5.0 | |
| Osmolarity | 900 | mOsm/L |

### Example H

| Vancomycin hydrochloride | 5 | mg/mL |
|---|---|---|
| D-lactic acid | 100 | mM |
| Propylene glycol | 800 | mM |
| Methionine | 10 | mM |
| pH | 5.0 | |
| Osmolarity | 1000 | mOsm/L |

### Example I

| | |
|---|---|
| Vancomycin hydrochloride | 5 mg/mL |
| Sodium D-lactate | 100 mM |
| Propylene glycol | 150 mM |
| Glycerol | 450 mM |
| pH | 5.0 |
| Osmolarity | 800 mOsm/L |

### Example J

| | |
|---|---|
| Vancomycin hydrochloride | 5 mg/mL |
| D-lactic acid | 100 mM |
| Propylene glycol | 150 mM |
| Sucrose | 350 mM |
| pH | 5.0 |
| Osmolarity | 690 mOsm/L |

### Example K

| | |
|---|---|
| Vancomycin hydrochloride | 5 mg/mL |
| Sodium D-lactate | 200 mM |
| Mannitol | 400 mM |
| pH | 5.0 |
| Osmolarity | 800 mOsm/L |

### Example L

| Vancomycin hydrochloride | 5 | mg/mL |
|---|---|---|
| D-lactic acid | 100 | mM |
| Propylene glycol | 1000 | mM |
| Sodium chloride | 10 | mM |
| pH | 5.0 | |
| Osmolarity | 1210 | mOsmol/L |

### Example M

| | |
|---|---|
| Vancomycin hydrochloride | 5 mg/mL |
| D-lactic acid | 100 mM |
| Sucrose | 200 mM |
| Propylene glycol | 300 mM |
| Sodium chloride | 100 mM |
| pH | 5.0 |
| Osmolarity | 890 mOsm/L |

In all of the above compositions the pH of the composition is adjusted to the required pH by the addition of a base such as sodium hydroxide. It should be noted that in cases where “sodium chloride” is listed as a composition component, the stated amount of sodium chloride is added to the composition.

### Example 2: Effect of pH on Stability of Vancomycin Compositions

The effect of pH on the stability of vancomycin compositions following storage for 3 days at 60° C. was investigated using the RP-HPLC described in General Methods. All compositions contained 5 mg/mL vancomycin hydrochloride in water for injection (WFI). The results are shown in Table 1 which shows % impurities observed in the compositions at time zero (TO) and after storage for 3 days at 60° C.

TABLE 1
Stability of vancomycin hydrochloride (5 mg/mL)
compositions assessed using RP-HPLC

% Impurities

| Composition No. | pH | T0 | 3 days (60° C.) |
|---|---|---|---|
| 1 | 3.5 | 5.57% | 49.48% |
| 2 | 4.5 | 5.56% | 44.59% |
| 3 | 5.5 | 5.69% | 43.98% |
| 4 | 6.5 | 5.77% | 45.99% |
| 5 | 7.2 | 5.78% | 52.13% |
| 6 | 9.5 | 5.97% | 77.64% |

It can be seen from Table 1 that the pH range 4.5-6.5 was the most stable, with the degradation rate accelerating either side of these ranges and being much quicker at more alkaline pH. All compositions in subsequent examples were prepared at pH 5.0.

### Example 3: Effect of Composition Excipients on Stability of Vancomycin

The effect of individual composition excipients N-acetyl-D-alanine (NADA), PEG400 and lysine hydrochloride on the stability of vancomycin was investigated using the RP-HPLC method described in General Methods. All compositions comprised 5 mg/mL vancomycin hydrochloride in water for injection (WFI) and the additional excipients as set out in Table 2 (below), and were adjusted to pH 5.0. Table 2 shows % impurities observed in the compositions at time zero (TO) and after storage for 15 and 30 days at 25° C. Composition 7 corresponds to the marketed composition of VancoReady™.

TABLE 2
Stability of vancomycin hydrochloride (5 mg/mL) compositions
expressed as % impurities vs. time zero (T0)

| | | | Lysine | | % Impurities | | |
|---|---|---|---|---|---|---|---|
| Composition | NADA | PEG400 | HCl | Osmolarity | 0 days | 15 days | 30 days |
| No. | (mM) | (mM) | (mM) | (mOsm/L) | (25° C.) | (25° C.) | (25° C.) |
| 7* | 103.7 | 50.8 | 69 | 292.5 | 3.60 | 3.68 | 4.08 |
| 8 | — | 50.8 | 69 | 188.8 | 5.31 | 12.20 | 17.76 |
| 9 | 103.7 | 50.8 | — | 154.5 | 5.10 | 5.46 | 5.97 |
| 10 | 103.7 | — | 69 | 241.7 | 4.74 | 5.14 | 5.01 |

*(VancoReady ™ composition)

It can be seen from Table 2 that removal of NADA from the VancoReady™ product resulted in a significant impairment in stability (comparing compositions 7 and 8). Removal of PEG400 or lysine HCl appeared to have no significant impact on the stability of the vancomycin compositions. As such, NADA appears to be the key stabilizing excipient in VancoReady™.

### Example 4: Stability of Vancomycin Compositions in the Presence of Various Organic Acids and Propylene Glycol

The effect of various excipients (N-acetyl-D-alanine (NADA), N-acetyl-L-alanine (NALA), PEG400, lysine hydrochloride, sodium D-lactate, sodium L-lactate and propylene glycol (PG)) on the stability of vancomycin compositions was investigated using the RP-HPLC method described in General Methods. All compositions comprised 5 mg/ml vancomycin hydrochloride in water for injection (WFI) and the additional excipients as set out in Table 3 (below), and were adjusted to pH 5.0. Table 4 shows % impurities observed in the compositions at time zero (TO) and after storage for 6, 12, 40, and 69 days at 25° C. Composition 7 corresponds to the marketed composition of VancoReady™.

TABLE 3
Vancomycin hydrochloride (5 mg/mL) compositions

| | | | | Lysine | D- | L- | | |
|---|---|---|---|---|---|---|---|---|
| Composition | NADA | NALA | PEG400 | HCl | lactate | lactate | PG | Osmolarity |
| No. | (mM) | (mM) | (mM) | (mM) | (mM) | (mM) | (mM) | (mOsm/L) |
| 7* | 103.7 | — | 50.8 | 69 | — | — | — | 293 |
| 8 | — | — | 50.8 | 69 | — | — | — | 189 |
| 13 | — | 103.7 | 50.8 | 69 | — | — | — | 293 |
| 14 | — | — | 50.8 | 69 | 103.7 | — | — | 396 |
| 15 | — | — | — | 69 | 103.7 | — | 2124 | 2469 |
| 16 | — | — | 50.8 | 69 | — | 103.7 | — | 396 |
| 17 | — | — | — | 69 | — | 103.7 | 2124 | 2469 |

*(VancoReady ™ composition)

TABLE 4
Stability of vancomycin hydrochloride (5 mg/mL)
compositions assessed using RP-HPLC

| | % Impurities | | | | |
|---|---|---|---|---|---|
| Composition | 0 days | 6 days | 12 days | 40 days | 69 days |
| No. | (25° C.) | (25° C.) | (25° C.) | (25° C.) | (25° C.) |
| 7* | 3.46% | 3.64% | 3.71% | 4.04% | 4.46% |
| 8 | 4.59% | 7.85% | 10.55% | 20.85% | NT** |
| 13 | 4.55% | 7.90% | 11.00% | 20.79% | NT** |
| 14 | 4.55% | 5.92% | 7.07% | 11.10% | 15.37% |
| 15 | 4.38% | 5.14% | 5.85% | 8.77% | 11.35% |
| 16 | 4.55% | 7.57% | 10.15% | 19.33% | NT** |
| 17 | 4.47% | 6.37% | 8.30% | 16.00% | 23.53% |

*(VancoReady ™ composition)
**(Analysis not performed due to sample failing visual inspection due to precipitation of the formulation)

Comparing compositions 7 and 8, again it can be seen that removal of NADA from the VancoReady™ composition results in a significant impairment in stability. Replacing NADA with N-acetyl-L-alanine (NALA) also resulted in a significant impairment in stability (comparing compositions 7 and 13). However, when NADA was replaced with sodium D-lactate, the reduction in composition stability was considerably smaller (comparing compositions 7 and 14). The same effect was not observed for sodium L-lactate, where stability was considerably reduced (comparing compositions 7 and 16). Modifying the compositions containing sodium D-lactate and sodium L-lactate to replace the PEG400 with the polyol propylene glycol, a surprising relative increase in stability was observed (comparing compositions 14 and 15, and compositions 16 and 17).

### Example 5: Stability of Vancomycin Compositions in the Presence of Higher Concentrations of D-Lactate and Propylene Glycol, and in the Presence of Inorganic Salts

The effect of varying concentrations of sodium D-lactate and propylene glycol (PG), and the effect of adding inorganic salts to vancomycin compositions was investigated using the RP-HPLC method described in General Methods. All compositions comprised 5 mg/ml vancomycin hydrochloride in water for injection (WFI) and the additional amounts excipients as set out in Table 5 (below) and were adjusted to pH 5.0. Table 6 shows the increase in % total impurities (compared with time zero) following storage for 2, 4, and 8 weeks at 25° C.

TABLE 5
Vancomycin hydrochloride (5 mg/mL) compositions

| Composition | D-lactate | PG | Sodium Chloride | Osmolarity |
|---|---|---|---|---|
| No. | (mM) | (mM) | (mM) | (mOsm/L) |
| 18 | 103.7 | 2124 | — | 2331 |
| 19 | 300 | 2124 | — | 2724 |
| 20 | 103.7 | 965 | — | 1172 |
| 21 | 103.7 | 2124 | 150 | 2631 |

TABLE 6
Stability of vancomycin hydrochloride (5 mg/mL)
compositions assessed using RP-HPLC

| | Increase in % total Impurities (compared with time zero) | | |
|---|---|---|---|
| Composition | 2 weeks | 4 weeks | 8 weeks |
| No. | (25° C.) | (25° C.) | (25° C.) |
| 18 | 1.57% | 2.47% | 5.15% |
| 19 | 0.73% | 0.92% | 2.25% |
| 20 | 1.88% | 3.24% | 6.34% |
| 21 | 1.59% | 2.53% | 5.03% |

It can be seen from Table 6 that increasing the concentration of sodium D-lactate leads to an improvement in composition stability (comparing compositions 18 and 19), as does increasing the concentration of propylene glycol (comparing compositions 20 and 21). The addition of inorganic salt sodium chloride provides a further stabilizing effect (comparing compositions 18 and 21).

### Example 6: Stability of Vancomycin Compositions (5 mg/mL) in the Presence of Varying Concentrations of D-Lactate, DL-Lactate and Propylene Glycol, and Further Excipients

The effect of varying concentrations of sodium D-lactate, sodium DL-lactate and propylene glycol (PG) on the stability of vancomycin compositions was investigated using the RP-HPLC method described in General Methods. The effect of adding sodium oxamate, polysorbate 80 and methionine was also investigated. All compositions comprised 5 mg/mL vancomycin hydrochloride in water for injection (WFI) and the additional amounts excipients as set out in Table 7 (below), and were adjusted to pH 5.0. Table 8 shows the increase in % total impurities (compared with time zero) following storage for 8 weeks at 2-8° C., and for 8 weeks at 25° C.

TABLE 7
Vancomycin hydrochloride (5 mg/mL) compositions

| | D- | DL- | | Sodium | Polysorbate | | |
|---|---|---|---|---|---|---|---|
| Composition | lactate | lactate | PG | Oxamate | 80 | Methionine | Osmolarity |
| No. | (mM) | (mM) | (mM) | (mM) | (mg/mL) | (mM) | (mOsm/L) |
| 23 | 150 | — | 486 | — | — | — | 786 |
| 24 | 150 | — | 959 | — | — | — | 1259 |
| 25 | 150 | — | 1932 | — | — | — | 2232 |
| 26 | 300 | — | 486 | — | — | — | 1086 |
| 27 | 300 | — | 959 | — | — | — | 1559 |
| 28 | 300 | — | 1932 | — | — | — | 2532 |
| 29 | — | 300 | 959 | — | — | — | 1559 |
| 30 | — | 600 | 959 | — | — | — | 2159 |
| 31 | 150 | — | 959 | 100 | — | — | 1459 |
| 32 | 150 | — | 1932 | — | — | 30 | 2262 |
| 33 | 150 | — | 959 | — | 2 | — | 1259 |
| 34 | 150 | — | 1932 | — | 2 | — | 2232 |

TABLE 8
Stability of vancomycin hydrochloride (5 mg/mL)
compositions assessed using RP-HPLC

| Composition | Increase in % total Impurities (compared with time zero) | |
|---|---|---|
| No. | 8 weeks (2-8° C.) | 8 weeks (25° C.) |
| 23 | 0.39% | 5.79% |
| 24 | 0.37% | 5.27% |
| 25 | 0.29% | 4.34% |
| 26 | 0.28% | 3.35% |
| 27 | 0.22% | 3.11% |
| 28 | 0.22% | 2.46% |
| 29 | 0.36% | 4.90% |
| 30 | 0.27% | 2.70% |
| 31 | 0.29% | 3.55% |
| 32 | 0.26% | 3.11% |
| 33 | 0.28% | 5.02% |
| 34 | 0.23% | 2.90% |

It can be seen from Table 8 that increasing the concentration of sodium D-lactate results in improved composition stability (comparing compositions 23 and 26, 24 and 27, and 25 and 28). Increasing the concentration of PG also results in improved composition stability (comparing compositions 23-24, and compositions 26-28). Interestingly, the racemic form of lactate (sodium DL-lactate) also provides a stabilizing effect, but the concentration must be twice as high as if D-lactate is used for a comparable stabilizing effect (comparing compositions 28-30).

The addition of sodium oxamate leads to an improvement in stability (comparing compositions 24 and 31). The addition of methionine results in a small improvement of stability (comparing compositions 25 and 32), as does the addition of polysorbate 80 (comparing compositions 24 and 33, and 25 and 34).

### Example 7: Stability of Vancomycin Compositions (50 mg/mL) in the Presence of Varying Concentrations of D-Lactate, DL-Lactate and Polyol, and Sodium Chloride

The effect of varying concentrations of sodium D-lactate, sodium DL-lactate and polyol (propylene glycol (PG) or glycerol) on the stability of vancomycin compositions was investigated using the RP-HPLC method described in General Methods. The effect of adding sodium chloride was also investigated. All compositions comprised 50 mg/mL vancomycin hydrochloride in water for injection (WFI) and the additional amounts excipients as set out in Table 9 (below), and were adjusted to pH 5.0. Table 10 shows the increase in % total impurities (compared with time zero) following storage for 8 weeks at 2-8° C., and for 8 weeks at 25° C.

TABLE 9
Vancomycin hydrochloride (50 mg/mL) compositions

| Compo- | D- | DL- | | | Sodium | |
|---|---|---|---|---|---|---|
| sition | lactate | lactate | PG | Glycerol | Chloride | Osmolarity |
| No. | (mM) | (mM) | (mM) | (mM) | (mM) | (mOsm/L) |
| 23 | 200 | — | 1971 | — | — | 2371 |
| 24 | 200 | — | 6571 | — | — | 6971 |
| 25 | 200 | — | 4600 | 1629 | — | 6629 |
| 26 | 450 | — | 1971 | — | — | 2871 |
| 27 | 450 | — | — | 1629 | — | 2529 |
| 28 | — | 400 | 6571 | — | — | 7371 |
| 29 | 200 | — | 1971 | — | 100 | 2571 |

TABLE 10
Stability of vancomycin hydrochloride (50
mg/mL) compositions assessed using RP-HPLC

| | |
|---|---|
| Composition | Δ Total Impurities (%) |

| No. | 8 weeks (2-8° C.) | 8 weeks (25° C.) |
|---|---|---|
| 23 | NT* | NT* |
| 24 | 0.14% | 0.56% |
| 25 | 0.15% | 0.53% |
| 26 | 0.11% | 0.79% |
| 27 | 0.17% | 0.96% |
| 28 | 0.13% | 0.69% |
| 29 | 0.15% | 1.42% |

*Analysis not performed due to sample failing visual inspection due to precipitation and formation of a gel

It can be seen from Table 10 that increasing the concentration of sodium D-lactate results in improved composition stability (comparing compositions 23 and 26). Increasing the concentration of polyol also results in improved composition stability (comparing compositions 23 and 24). Interestingly, the racemic form of lactate (sodium DL-lactate) also provides a stabilizing effect, but the concentration must be twice as high as if D-lactate is used for a comparable stabilizing effect (comparing compositions 24 and 28).

The addition of sodium chloride leads to an improvement in stability (comparing compositions 23 and 29). A mixture of polyols (propylene glycol and glycerol) was also found to have a stabilizing effect (comparing compositions 24 and 25).

### Example 8: Stability of Vancomycin Compositions (50 mg/mL) in the Presence of Inorganic Salts Containing a Metal Cation

The effect of adding sodium chloride and calcium chloride on the stability of vancomycin compositions was investigated using the Visual Assessment Method described in General Methods. All compositions comprised 50 mg/mL vancomycin hydrochloride in water for injection (WFI), sodium D-lactate and polyol (propylene glycol and glycerol mixture) in the amounts shown in Table 11 (below), and were adjusted to pH 5.0. Table 12 shows the visual appearance of the compositions following storage for 19 weeks at 25° C.

TABLE 11
Vancomycin hydrochloride (50 mg/mL) compositions

| Compo- | D- | | | Sodium | Calcium | |
|---|---|---|---|---|---|---|
| sition | lactate | PG | Glycerol | Chloride | chloride | Osmolarity |
| No. | (mM) | (mM) | (mM) | (mM) | (mM) | (mOsm/L) |
| 30 | 200 | 1971 | 1086 | — | — | 3457 |
| 31 | 200 | 1971 | 1086 | 300 | — | 3757 |
| 32 | 200 | 1971 | 1086 | — | 300 | 3757 |
| 33 | 400 | 1971 | 1086 | — | — | 3857 |
| 34 | 400 | 1971 | 1086 | 300 | — | 4157 |

TABLE 12
Stability of vancomycin hydrochloride (50 mg/mL)
compositions assessed using visual assessment

Visual assessment

| Composition | Visual assessment | Visual assessment |
|---|---|---|
| No. | (19 weeks at 25° C.) | (19 weeks at 2-8° C.) |
| 30 | 1 | 5 |
| 31 | 1 | 1 |
| 32 | 1 | 1 |
| 33 | 2 | 5 |
| 34 | 1 | 1 |

It can be seen from Table 12 the addition of both sodium chloride and calcium chloride provided a significant improvement in stability of the compositions when stored at 2-8° C.

### Example 9: Stability of Vancomycin Compositions (5 mg/mL) in the Presence of a Range of Polyols

The effect of selected polyols (propylene glycol (PG), sucrose, trehalose, glycerol and mannitol) in the presence of 103.7 mM D-lactate on the stability of vancomycin compositions was investigated using the RP-HPLC method described in General Methods. All compositions comprised 5 mg/mL vancomycin hydrochloride in water for injection (WFI) and the additional excipients as set out in Table 13 (below), and were adjusted to pH 5.0. Table 14 shows the increase in % total impurities (compared with time zero) after storage for 2, 4 and 6 weeks at 25° C.

TABLE 13
Vancomycin hydrochloride (5 mg/mL) compositions

| | | D- | | | | | |
|---|---|---|---|---|---|---|---|
| Composition | PG | lactate | Sucrose | Trehalose | Glycerol | Mannitol | Osmolarity |
| No. | (mM) | (mM) | (mM) | (mM) | (mM) | (mM) | (mOsm/L) |
| 35 | 965 | 103.7 | — | — | — | — | 1172.4 |
| 36 | 330 | 103.7 | — | — | — | — | 537.4 |
| 37 | — | 103.7 | 330 | — | — | — | 537.4 |
| 38 | — | 103.7 | — | 330 | — | — | 537.4 |
| 39 | — | 103.7 | — | — | 330 | — | 537.4 |
| 40 | — | 103.7 | — | — | — | 330 | 537.4 |

TABLE 14
Stability of vancomycin hydrochloride (5 mg/mL)
compositions assessed using RP-HPLC

| | Δ Total Impurities (%) | | |
|---|---|---|---|
| Composition | 2 weeks | 4 weeks | 8 weeks |
| No. | (25° C.) | (25° C.) | (25° C.) |
| 35 | 1.88 | 3.24 | 6.34 |
| 36 | 2.25 | 3.77 | 7.54 |
| 37 | 2.18 | 3.75 | 7.48 |
| 38 | 2.15 | 3.62 | 7.19 |
| 39 | 2.28 | 3.88 | 7.82 |
| 40 | 2.29 | 3.89 | 7.83 |

Comparing compositions 36 to 40, we can observe that the polyols tested have similar effects on stability at equimolar concentrations tested. Further, comparing formulations 36 to 40, to formulation 35, we observe that increased concentration of polyol results in improved stability at the expense of increased osmolarity. It is therefore necessary to identify optimal balance between stability and osmolarity depending on product requirement.

### Example 10: Long-Term Stability of Ready-to-Administer Vancomycin Compositions (5 mg/mL)

Long-term stability trials for three compositions were set up at 2-8° C. and 25° C. All compositions comprised 5 mg/mL vancomycin hydrochloride and additional excipients shown in Table 15 and were adjusted to pH 5.0. Compositions were filled, under aseptic conditions, into Technoflex polypropylene 50 mL IV bags (Technoflex IX01001T009.029_D). The stability of the compositions at 2-8° C. and 25° C. was investigated using the RP-HPLC and Visual Assessment methods described in General Methods. All compositions remained clear and colourless (Visual score 1) throughout the long-term stability trials. The RP-HPLC results, in particular the increase in % total, and specific individual, impurities are shown in Tables 16-18 (2-8° C.) and 19-21 (25° C.).

TABLE 15
Vancomycin hydrochloride (5 mg/mL) compositions

| | Sodium D/ | Sodium D- | | | Sodium |
|---|---|---|---|---|---|
| Composition | L-lactate | lactate | PG | Glycerol | Chloride |
| No. | (mM) | (mM) | (mM) | (mM) | (mM) |
| 41 | — | 150 | 700 | — | 70 |
| 42 | — | 103.7 | — | 330 | — |
| 43 | 207.4 | — | — | 330 | — |

TABLE 16
Stability of Composition No. 41, following storage at 2-8° C., assessed using RP-HPLC

| | Total | Impurity | Impurity | Impurity | Impurity | Impurity | Other |
|---|---|---|---|---|---|---|---|
| Time | impurities | A | B1 | B2 | C | D | Impurities |
| (months) | (%) | (%) | (%) | (%) | (%) | (%) | (%) |
| 0 | 3.85 | 0.87 | 1.03 | 0.08 | 0.08 | 0.21 | 1.58 |
| 1 | 3.86 | 0.85 | 0.97 | 0.00 | 0.00 | 0.21 | 1.83 |
| 2 | 4.36 | 0.73 | 1.85 | 0.23 | 0.00 | 0.17 | 1.38 |
| 3 | 4.46 | 1.00 | 1.29 | 0.82 | 0.00 | 0.11 | 1.24 |
| 6 | 5.14 | 0.75 | 1.20 | 0.55 | 0.00 | 0.19 | 2.45 |

TABLE 17
Stability of Composition No. 42, following storage at 2-8° C., assessed using RP-HPLC

| | Total | Impurity | Impurity | Impurity | Impurity | Impurity | Other |
|---|---|---|---|---|---|---|---|
| Time | impurities | A | B1 | B2 | C | D | Impurities |
| (months) | (%) | (%) | (%) | (%) | (%) | (%) | (%) |
| 0 | 3.91 | 0.89 | 0.98 | 0.08 | 0.08 | 0.20 | 1.68 |
| 1 | 3.93 | 0.87 | 0.99 | 0.00 | 0.00 | 0.20 | 1.87 |
| 2 | 3.75 | 0.71 | 1.40 | 0.22 | 0.00 | 0.15 | 1.27 |
| 3 | 4.76 | 1.10 | 1.31 | 0.85 | 0.00 | 0.12 | 1.38 |
| 6 | 5.42 | 0.83 | 1.26 | 0.74 | 0.00 | 0.19 | 2.40 |

TABLE 18
Stability of Composition No. 43, following storage at 2-8° C., assessed using RP-HPLC

| | Total | Impurity | Impurity | Impurity | Impurity | Impurity | Other |
|---|---|---|---|---|---|---|---|
| Time | impurities | A | B1 | B2 | C | D | Impurities |
| (months) | (%) | (%) | (%) | (%) | (%) | (%) | (%) |
| 0 | 3.97 | 0.88 | 0.99 | 0.09 | 0.08 | 0.21 | 1.72 |
| 1 | 3.8 | 0.87 | 1.00 | 0.00 | 0.00 | 0.20 | 1.73 |
| 2 | 4.32 | 0.70 | 1.83 | 0.26 | 0.00 | 0.15 | 1.38 |
| 3 | 4.71 | 1.07 | 1.30 | 0.86 | 0.00 | 0.12 | 1.36 |
| 6 | 4.77 | 0.85 | 1.23 | 0.70 | 0.00 | 0.19 | 1.80 |

TABLE 19
Stability of Composition No. 41, following storage at 25° C., assessed using RP-HPLC

| | Total | Impurity | Impurity | Impurity | Impurity | Impurity | Other |
|---|---|---|---|---|---|---|---|
| Time | impurities | A | B1 | B2 | C | D | Impurities |
| (months) | (%) | (%) | (%) | (%) | (%) | (%) | (%) |
| 0 | 3.85 | 0.87 | 1.03 | 0.08 | 0.08 | 0.21 | 1.58 |
| 0.5 | 4.87 | 1.03 | 1.29 | 0.79 | 0.00 | 0.18 | 1.58 |
| 2 | 7.74 | 0.53 | 2.59 | 3.19 | 0.00 | 0.11 | 1.32 |

TABLE 20
Stability of Composition No. 42, following storage at 25° C., assessed using RP-HPLC

| | Total | Impurity | Impurity | Impurity | Impurity | Impurity | Other |
|---|---|---|---|---|---|---|---|
| Time | impurities | A | B1 | B2 | C | D | Impurities |
| (months) | (%) | (%) | (%) | (%) | (%) | (%) | (%) |
| 0 | 3.91 | 0.89 | 0.98 | 0.08 | 0.08 | 0.20 | 1.68 |
| 0.5 | 5.38 | 1.19 | 1.40 | 1.00 | 0.00 | 0.17 | 1.62 |
| 2 | 10.17 | 0.49 | 3.39 | 4.82 | 0.00 | 0.17 | 1.30 |

TABLE 21
Stability of Composition No. 43, following storage at 25° C., assessed using RP-HPLC

| | Total | Impurity | Impurity | Impurity | Impurity | Impurity | Other |
|---|---|---|---|---|---|---|---|
| Time | impurities | A | B1 | B2 | C | D | Impurities |
| (months) | (%) | (%) | (%) | (%) | (%) | (%) | (%) |
| 0 | 3.97 | 0.88 | 0.99 | 0.09 | 0.08 | 0.21 | 1.72 |
| 0.5 | 5.14 | 1.09 | 1.36 | 0.94 | 0.00 | 0.17 | 1.58 |
| 2 | 9.5 | 0.53 | 3.20 | 4.40 | 0.00 | 0.19 | 1.18 |

Significantly, the US Pharmacopeial Monograph (Vancomycin hydrochloride for Injection, USP29) requires that a vancomycin drug product has the following purity:
No less than 88% vancomycin purity (i.e. no more than 12% total impurities).
No more than 4% of any individual impurity

The results in Table 16-21 demonstrate that the trialed compositions (Compositions No. 41-43) are long-term storage stable, and in particular are in compliance with the conditions set out in the US Pharmacopeial Monograph (USP29), upon storage at 2-8° C. for 6 months and following a short-term excursion at 25° C.

These results indicate that the trialed compositions (Compositions No. 41-43) are expected to have a shelf-life of at least 24 months at 2-8° C., and that said compositions are expected to remain stable, and furthermore remain compliant with the conditions set out in the US Pharmacopeial Monograph (USP29) following a short-term excursion at 25° C.

1. A storage stable aqueous solution composition comprising:
vancomycin or a pharmaceutically acceptable salt thereof, at a concentration of 1-10 mg/mL;
D-lactic acid or a pharmaceutically acceptable salt thereof; and
a polyol selected from the group consisting of propylene glycol, sucrose, glycerol, trehalose, lactose, glucose, sorbitol and mannitol, or a mixture thereof;
wherein the osmolarity of the composition is 400-1250 mOsm/L.

2. The storage stable aqueous solution composition according to claim 1,
wherein the vancomycin form is vancomycin hydrochloride; and/or
wherein the concentration of vancomycin or a pharmaceutically acceptable salt thereof is 2.5-7.5 mg/mL.

3. The storage stable aqueous solution composition according to claim 1, wherein the concentration of D-lactic acid or a pharmaceutically acceptable salt thereof is 100-500 mM.

4. The storage stable aqueous solution composition according to claim 1, wherein the source of the D-lactic acid or a pharmaceutically acceptable salt thereof is DL-lactic acid or a pharmaceutically acceptable salt thereof.

5. The storage stable aqueous solution composition according to claim 4, wherein the concentration of DL-lactic acid or a pharmaceutically acceptable salt thereof is 200-1000 mM.

6. The storage stable aqueous solution composition according to claim 1, wherein the total concentration of polyol in the composition is 200-1200 mM.

7. The storage stable aqueous solution composition according to claim 1, wherein the composition comprises a single polyol selected from the group consisting of propylene glycol, sucrose, glycerol, trehalose, lactose, glucose, sorbitol and mannitol.

8. The storage stable aqueous solution composition according to claim 1, wherein the composition comprises a mixture of two polyols selected from the group consisting of propylene glycol, sucrose, glycerol, trehalose, lactose, glucose, sorbitol and mannitol.

9. The storage stable aqueous solution composition according to claim 1, wherein the or each polyol is selected from the group consisting of sucrose, glycerol, trehalose, lactose, glucose, sorbitol and mannitol.

10. The storage stable aqueous solution composition according to claim 1, wherein the or each polyol is selected from the group consisting of propylene glycol, sucrose, trehalose, lactose, glucose, sorbitol and mannitol.

11. The storage stable aqueous solution composition according to claim 1, wherein the or each polyol is selected from the group consisting of sucrose, trehalose, lactose, glucose, sorbitol and mannitol.

12. The storage stable aqueous solution composition according to claim 8, wherein the composition comprises a mixture of propylene glycol and a polyol selected from the group consisting of sucrose, glycerol, trehalose, lactose, glucose, sorbitol and mannitol; wherein the propylene glycol is present at a concentration of 10-160 mM; and the polyol selected from the group consisting of sucrose, glycerol, trehalose, lactose, glucose, sorbitol and mannitol is present at a concentration of 40-1190 mM.

13. The storage stable aqueous solution composition according to claim 12, wherein the combined total concentration of propylene glycol and polyol selected from the group consisting of sucrose, glycerol, trehalose, lactose, glucose, sorbitol and mannitol is 200-1200 mM.

14. The storage stable aqueous solution composition according to claim 12, wherein the composition comprises a mixture of propylene glycol and glycerol, wherein the propylene glycol is present in the composition at a concentration of 10-160 mM, and the glycerol is present in the composition at a concentration of 40-1190 mM, and wherein the combined total concentration of propylene glycol and glycerol is 200-1200 mM.

15. The storage stable aqueous solution composition according to claim 12, wherein the composition comprises a mixture of propylene glycol and sucrose, wherein the propylene glycol is present in the composition at a concentration of 10-160 mM, and the sucrose is present in the composition at a concentration of 40-1190 mM; and wherein the combined total concentration of propylene glycol and sucrose is 200-1200 mM.

16. The storage stable aqueous solution composition according to claim 12, wherein the composition comprises a mixture of propylene glycol and mannitol, wherein the propylene glycol is present in the composition at a concentration of 10-160 mM, and the mannitol is present in the composition at a concentration of 40-1190 mM; and wherein the combined total concentration of propylene glycol and mannitol is 200-1200 mM.

17. The storage stable aqueous solution composition according to claim 1, wherein the molar ratio of vancomycin or a pharmaceutically acceptable salt thereof, to polyol is between 1:100 and 1:1000.

18. The storage stable aqueous solution composition according to claim 1, wherein the osmolarity of the composition is 410-1250 mOsm/L.

19. The storage stable aqueous solution composition according to claim 1, comprising an inorganic salt containing a metal cation, at a concentration of 1-500 mM.

20. The storage stable aqueous solution composition according to claim 1, wherein the pH of the composition is in the range 4.0 to 6.0.

21. The storage stable aqueous solution composition according to claim 1, comprising at least 2.5% (v/v) water.

22. The storage stable aqueous solution composition according to claim 1, which is a pharmaceutical composition.

23. (canceled)

24. (canceled)

25. A ready-to-administer intravenous (IV) solution container containing the storage stable aqueous solution composition according to claim 1.

26. A method of treating bacterial infection which comprises administering to a patient in need thereof a therapeutically effective amount of a storage stable aqueous solution composition according to claim 1.

27. The method according to claim 26, wherein the bacterial infection is caused by gram positive bacteria, and is selected from the group consisting of Clostridium difficile, Listeria monocytogenes, Streptococcus pyogenes, Streptococcus pneumoniae, Streptococcus agalactiae, Streptococcus viridans, Streptococcus bovis, Staphylococcus epidermidis, multidrug resistant Staphylococcus epidermidis, Staphylococcus aureus, methicillin-resistant Staphylococcos aureus, Actinomyces species, Lactobacillus species, Enterococcus faecalis, and diphtheroids.
